(19) United States
(12) Patent Application Publication
(10) Pub. No.: US 2009/0092550 A1
(43) Pub. Date: Apr. 9, 2009

(54) PORPHYRIN LINKED METRONIDAZOLE AGAINST GUM DISEASE: PORPHYROMONAS GINGIVALIS

(75) Inventors: Max Crossley, Beecroft (AU); Pall Thordarson, Coogee (AU); Neil Hunter, Pennant Hills (AU); Benjamin Yap, Chippendale (AU); Charles Andrew Collyer, Glebe (AU)

Correspondence Address: KNOBBE MARTENS OLSON & BEAR LLP, 2040 MAIN STREET, FOURTEENTH FLOOR, IRVINE, CA, 92614, US

(73) Assignee: The University of Sydney, Camperdown (AU)

(21) Appl. No.: 11/572,102

(22) PCT Filed: Jul. 15, 2005
(86) PCT No.: PCT/AU2005/001038

§ 371 Date: Sep. 5, 2007

Related U.S. Application Data
(60) Provisional application No. 60/588,401, filed on Jul. 15, 2004.

(30) Foreign Application Priority Data
Jul. 15, 2004 (AU) 2004903943

(51) Int. Cl. A61K 49/00; C07D 245/00; A61K 31/40

(52) U.S. Cl. 424/9.1; 540/471; 514/410

(57) ABSTRACT

The present invention relates generally to targeted molecular agents (TMAs) directed to a particular organism or group of organisms and uses thereof. More particularly, the present invention provides TMAs having a targeting moiety which comprises a natural or induced auxotrophic requirement of the particular organism as a vehicle for directing an agent linked to the moiety to be delivered to the target organism. The TMAs of the present invention are useful for targeting molecules such as antimicrobial agents and diagnostic agents to selected organisms.

### BACKGROUND OF THE INVENTION

1. Field of the Invention

The present invention relates generally to targeted molecular agents (TMAs) directed to a particular organism or group of organisms and uses thereof. More particularly, the present invention provides TMAs having a targeting moiety which comprises a natural or induced auxotrophic requirement of the particular organism as a vehicle for directing an agent linked to the moiety to be delivered to the target organism. The TMAs of the present invention are useful for targeting molecules such as antimicrobial agents and diagnostic agents to selected organisms.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Infections of the tissues that support the teeth are a global problem for mammals. For example, in Australia, sheep are culled whilst still productive because of loosening and loss of teeth, a condition known as “broken-mouth periodontitis”. From the time of the earliest civilizations, there has been documentation of diseases affecting the periodontal tissues. Yet it is only in the last 40 years or so that indices of the periodontal diseases were developed and the bacterial etiology demonstrated. The incidence of periodontal diseases in adults has been reported to range from about 30% of the total population in the USA to about 70% of the population in developing countries.

Periodontal diseases are caused by a variety of factors that include local environmental factors such as inadequate oral hygiene, predisposing factors related to the morphology of the periodontium, hereditary factors, and modifying factors from systemic disease and periodontal trauma. Prevalence of periodontal disease has been reported to correlate with various socioeconomic factors such as level of education, income and ready access to dental treatment. Poorly controlled diabetes and smoking are also risk factors for the disease. The high prevalence of periodontal diseases and high economic cost associated with its treatment drives the imperative for more effective treatment.

Porphyromonas gingivalis resides in the tight confines of the deep periodontal pocket between the tooth and the gingival tissue (gingival crevice). The anatomy of the gingival crevice results in less effect of cleansing processes such as salivation and swallowing and this allows the occupation of microorganisms, such as P. gingivalis, to deliver their bacterial activity promoting gingivitis and over time leading to the progression of destructive periodontitis. In periodontal diseases, the redox potential in the subgingival area is low and the endogenous nutrients provided by the crevicular fluid flowing through the sulcus are rich in amino acids and peptides, thus providing an ideal environment for the colonization of P. gingivalis.

Porphyromonas gingivalis is one of the key pathogens implicated in destructive periodontitis. It is a Gram-negative anaerobic rod with a diameter of 0.3-0.5 μm. The distinctive features of P. gingivalis are the fimbriae, the capsule and vesicles.

The fimbriae (F) are thin and hair-like with a diameter of about 5 nm and are believed to mediate bacterial adhesion to the host tissues and coaggregation with early-colonizing bacteria within the subgingival microbiota. The capsule (C) is electron dense but its function is unknown. The vesicles (V) are circular and contain proteases required for the degradation of supporting tissue in the periodontal pocket and hydrolysis of host proteins to provide essential amino acids for growth of the organisms. The proteases also have the ability to disturb local immune responses and contribute in the processing and maturation of fimbrial proteins important in bacterial adhesion and coaggregation.

Heme is required by P. gingivalis for a number of different functions. Cell surface heme acquisition has been suggested to be an effective defense mechanism against active oxygen species and heme capture is likely to be important for energy metabolism. There are three putative genes, HemN, HemG and HemH, encoding the last three enzymes in the heme biosynthetic pathway present in the P. gingivalis genome. The intracellular expression of HemH (porphyrin ferrochelatase) permits exogenous protoporphyrin IX (PPIX), once captured and transported into the cell, to substitute as a growth factor for heme in an iron-replete environment by allowing the chelation of Fe²⁺ and PPIX into heme.

The presence of homologs of HemN (a coporphyrinogen oxidase) and HemG (a protoporphyrinogen oxidase) could also convey an ability to generate essential porphyrins from related porphyrins by modifications of functionality of the porphyrin macrocycle. The ability to use other iron sources in combination with exogenous non-iron porphyrins to bypass the growth requirement of heme suggests that at least some of P. gingivalis' porphyrin capture systems can recognize non-iron porphyrins. The gingipain Kgp is a hemoglobin protease and the HA2 domain is reported to bind heme and also functions as a hemophore to capture heme.

Hemoglobin is a ready source of heme-associated porphyrin and iron in the inflammed periodontal pocket and its binding in P. gingivalis has been clearly demonstrated by a TonB-dependent protein, HmuR and the HA2 domain of gingipains.

Metronidazole is an antibiotic known to show antibacterial activity against a wide range of anaerobes, including P. gingivalis. One major disadvantage of using metronidazole is its inability to differentiate between the different species of anaerobic bacteria present in the oral cavity and as a result is unable to selectively inhibit P. gingivalis.

As there is a large variety of bacteria present in the oral cavity that are both beneficial and harmful, the use of an antibiotic alone such as metronidazole provides no selectivity and would kill the entire anaerobic flora present.

Accordingly, there is a need for agents which are modified to enhance their selective targeting of particular organisms. In addition, this selectivity is useful as a general targeting vehicle for a range of molecules such as diagnostic agents or any other molecule required to be presented to a target organism.

### SUMMARY OF THE INVENTION

The present invention provides TMAs which are selective for particular organisms or group of organisms based on a particular auxotrophic requirement of the target organism. The auxotrophic requirement may be natural to the organism or artificially induced, such as by mutagenesis. The organism may be a prokaryotic microorganism or a eukaryotic organism including a parasite. For brevity the terms “organism” and “microorganism” may be used interchangeably. In a preferred embodiment, the present invention provides TMAs comprising porphyrin, a porphyrin analog or a porphyrin-like molecule targeting moiety which show specificity toward organisms which have an auxotrophic requirement for porphyrin or porphyrin-like molecules. Reference herein to “porphyrin” includes free-based porphyrins (i.e. non-metal containing porphyrins) as well as metalloporphyrins. A particularly preferred target organism is P. gingivalis and its relatives. In effect, the auxotrophic requirement provides a vehicle for delivery of any molecule linked thereto to a targeted organism or group of organisms. In the case of P. gingivalis, it is the uptake mechanism of the haemauxotrophic requirement which enables the specifity. In essence, the uptake mechanism for porphyrin may be metal-independent (e.g. HA2 system) or metal dependent (e.g. HasA system).

The present invention relates generally, therefore, to a TMA comprising the general Formula (I):

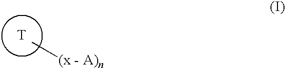

wherein T is a targeting moiety comprising an auxotrophic requirement of a target organism or an analog or derivative of said auxotrophic requirement, x is a chemical linkage entity or linker group, A is an agent required to be targeted to the organism and n is an integer greater than or equal to 1, wherein (x−A)₁, (x−A)₂, (x−A)₃ . . . (x−A)ₙ, may be the same or different and wherein each is independently linked to the targeting moiety by a chemical linkage entity.

The chemical linkage entity, may be the same for each (x−A)ₙ or may be different. Preferably, n=1. In any event, x may be an ester or amide or other form of chemical bond or linkage including a urea linkage or a carbamate linkage.

In another embodiment, the agent A is linked or complexed directly with the targeting moiety, T. In this instance the TMA comprises the general Formula (II):

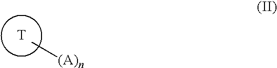

wherein T, A and n are defined as above.

Accordingly, the general formula may also be represented as Formula (III):

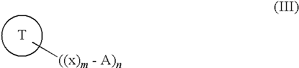

wherein T, x, A and n are defined as above and wherein m is 0 or 1.

The TMAs of the present invention have particular application for the treatment of infections in a biological environment by one or more organisms, which have one or more auxotrophic requirements. In this embodiment the targeted agent, A, is a cytotoxic molecule linked to the targeting moiety, T.

In a preferred embodiment, the targeting moiety, T, comprises porphyrin, a porphyrin analog or a porphyrin-like molecule linked to a cytotoxic agent. A porphyrin-like molecule includes a derivative porphyrin. As indicated above both free-based (i.e. non-metallo) porphyrins are metalloporphyrins by the present invention. In this embodiment, the TMA is used to treat infections in an animal, including human, subject by the organism P. gingivalis.

The present invention further extends to incorporation of the TMAs of the present invention in pharmaceutical or veterinary compositions and their use, inter alia, in treating microbial infection.

In another embodiment, the agent to be targeted is a reporter molecule used as a reporter molecule capable of providing an identifiable signal. Such a TMA would be regarded as a diagnostic agent which may be applied to, inter alia, the enumeration, localization or visualization of a particular organism or group of organisms.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

A list of abbreviations used herein is provided in Table 1.

TABLE 1
Abbreviations

| Abbreviation | Description |
|---|---|
| KD | Dissociation constant |
| TMA | Targeted Molecular Agent |
| ELISA | Enzyme Linked Immunosorbent Assay |
| DPIX | Deuteroporphyrin IX |
| MALDI-TOF | Matrix Assisted Laser Desorption Ionization- |
| | Time of Flight |
| TMS | Tetramethysilane |
| ESI | Electrospray ionization |
| TLC | Thin layer chromotography |
| NMR | Nuclear magnetic resonance |
| PPIX | Protoporphyrin IX |
| DME | Dimethyl ester |

### BRIEF DESCRIPTION OF THE FIGURES

FIG. 1 is a graphical representation showing a basic porphyrin structure and the numbering system (Fischer nomenclature). The terms α-pyrrolic and β-pyrrolic refer to the positions that are α- and β- to the nitrogen atoms of the pyrrolic rings of the porphyrin respectively. The bridging carbon atoms between the pyrrolic subunits refer to the meso positions and are labelled α, β, γ and δ. Letters A-D are used to represent the individual rings. The vinyl face of the porphyrin refers to carbons 1-4. The propionic acid face of the porphyrin refers to carbons 5-8.

FIG. 2 is a graphical representation showing the ¹H NMR spectrum of DPIX, where the singlet at 9.11 ppm is characteristic of the β-pyrrolic hydrogens at positions 2 and 4 and the singlets at 10.05, 10.10, and 10.17 ppm are characteristic of the four meso protons of the porphyrin macrocycle. The splitting of the singlet peak at 9.11 ppm in panel c) is due to coupling between the β-pyrrolic hydrogens and hydrogens from a neighbouring —CH₃ group. The triplets at 3.41-3.48 ppm and 4.43-4.50 ppm are due to the —CH₂ groups on the propanoic acid side chains and the singlets at 3.65, 3.68, 3.74, and 3.77 ppm are due to the four —CH₃ groups. Similarly, the small splitting of the singlet peaks at 3.74 and 3.77 ppm in panel e) are due to coupling between the —CH₃ protons with a neighbouring β-pyrrolic hydrogen. Finally, the peak at −4.13 ppm is characteristic of the inner NH protons, which further proves demetallation.

FIG. 3 is a graphical representation showing the ¹H NMR spectrum of DPIX di-substituted-metronidazole adduct 19.

FIG. 4 is a graphical representation showing the ¹H NMR spectrum of DPIX mono-substituted-metronidazole adducts 20 and 21.

FIG. 5 is a graphical representation showing the binding curve obtained as the plot of the optical density at 405 nm as a function of the concentration of HA2. KD stands for dissociation constant and KD50 is the concentration where half of the mono-adducts 20 and 21 is bound to HA2. Note that the association constant Ka is related to KD by the following equation: KD=1/Ka

FIG. 6 is a graphical representation showing the growth inhibition of P. gingivalis caused by compounds 20 and 21 at varying concentrations. A—Growth inhibition of P. gingivalis with 20 μM 20 and 21+ controls over 72 h; B—Growth inhibition of P. gingivalis with 4 μM 20 and 21+ controls over 72 h; C—Growth inhibition of P. gingivalis with 2 μM 20 and 21+ controls over 72 h; D—Growth inhibition of P. gingivalis with 1 μM 20 and 21+ controls over 72 h; E—Growth inhibition of P. gingivalis with 0.5 μM 20 and 21+ controls over 72 h.

FIG. 7 is a graphical representation showing the growth inhibition of P. gingivalis, Prevotella melaminogenica and Fusobacterium nucleatum by compounds 20 and 21 at 20 μM. A—Growth Inhibition of P. gingivalis with 20 μM 20 and 21+ controls over 72 h; B—Growth Inhibition of P. melaminogenica with 20 μM 20 and 21+ controls over 72 h; C—Growth Inhibition of F. nucleatum with 20 μM 20 and 21+ controls over 72 h.

### DETAILED DESCRIPTION OF THE PREFERRED EMBODIMENTS

The present invention provides a means of targeting an agent to a desired organism or group of organisms. The term “organism” includes prokaryotic and eukaryotic organisms including parasites. A prokaryotic organism includes a microorganism. Hence, the terms “organism” and “microorganism” may be used interchangeably in this specification without any limitation to the organism being a prokaryote or eukaryote. The targeting mechanism comprises a natural or induced auxotrophic requirement of the targeted organism as a vehicle to deliver any molecule or agent to an organism. In this instance, the organism may be of a single type, species, genus or family or may be a group of two or more types, species, genera or families of organisms having a common auxotrophic requirement. The targeting vehicle linked or otherwise associated with the molecule or agent to be delivered is referred to herein as a “targeted molecular agent” or TMA.

Before describing the present invention in detail, it is to be understood that unless otherwise indicated, the subject invention is not limited to specific formulations, synthesis methods, therapeutic protocols, or the like as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context already dictates otherwise. Thus, for example, reference to “a TMA” or “a targeted agent” includes a TMA or agent as well as two or more TMAs or agents, a “an auxotrophic requirement” includes a single requirement as well as two or more requirements; and so forth.

The present invention relates generally to a TMA comprising the general Formula (I):

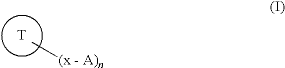

wherein T is a targeting moiety comprising an auxotrophic requirement of a target organism or an analog or derivative of said auxotrophic requirement, x is a chemical linkage entity or linker group, A is an agent required to be targeted to the organism and n is an integer greater than or equal to 1, wherein (x−A)₁, (x−A)₂, (x−A)₃ . . . (x−A)ₙ, may be the same or different and wherein each is independently linked to the targeting moiety via the chemical linkage entity or group, x, wherein in the case where n>1, x may be the same or different for each (x−A)ₙ entity.

The integer n may be any integer greater than or equal to 1, and includes the integers of 1, 2, 3, 4, 5, 6, 7, 8, 9, 10, 11, 12, 13, 14, 15, 16, 17, 18, 19 and 20. Preferably, however, n=1.

The representation

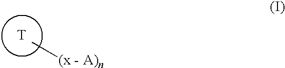

means that in effect T may be monovalent with respect to (x−A) or may be multivalent with multiple (x−A) entities, each of which may be the same or different. Furthermore, the chemical linkage entity may be placed anywhere on the T. x may be any type of bond but is preferably an ester or amide bond or a urea or carbamate linkage.

In certain circumstances, A may be linked, conjugated or otherwise associated with T without need for any linkage entity. In this case, the TMA comprises general Formula (II):

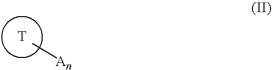

wherein T, A and n are as defined above.

Accordingly, in its broadest embodiment, the present invention provides a TMA comprising general Formula (III):

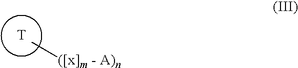

wherein T, X, A and n are defined as above and m is 0 or 1.

When T comprises multiple As, each of which is the same or different, then this is encompassed by Formula II

where

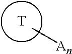

may be represent as

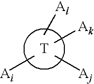

In this aspect, Aₙ=Aᵢ, Aⱼ, Aₖ, Aₗ . . . An where each of Aᵢ, Aⱼ . . . may be the same or different.

The representation in Formula II encompasses a linkage mechanism between each A and T.

The present invention includes isomers of the subject TMAs including monosulfonic and disulfonic derivatives of the TMAs compounds 20, 21, 39, 40, 41 and 42 described herein below are particularly useful including their isomers and mono- and disulfonic acid derivatives. 2-sulfonic acid and 4-sulfonic acid derivatives of compounds 20 and 21 are particularly preferred. Sulfonic acid groups can be incorporated of available positions on the porphyrin periphery (e.g. 2-, 4-, β-pyrrolic and α, β and δ meso positions).

As used herein, the term “auxotrophic requirement” refers to any nutrient, compound or growth factor that the organism must obligately sequester from its environment. This may be a natural requirement or an artificially induced requirement. As such, this term specifically includes any compound or nutrient which the organism is unable to synthesize itself. Exemplary auxotrophic requirements which in no way limit the invention include: essential amino acids, vitamins, heme and other porphyrins, organic growth factors, and the like.

The “agent required to be targeted to the organism”, set out as A in the general Formula (I) is also referred to herein as the “targeted agent”.

The TMAs of the present invention have particular application for the targeted cytotoxicity of an organism or group of organisms in a biological environment. Depending on the environment, this application may be referred to as a “treatment”, i.e. the selected reduction in the number of particular organisms. In this regard, the targeted agent is a cytotoxic molecule such as an antibiotic or other antimicrobial compound.

In another embodiment, the targeted agent is a reporter molecule for use such as in diagnosis or nucleic acid molecule such as for use in genetic manipulation.

In one preferred embodiment, however, the TMA is used to treat an infection in an animal including a human subject, wherein the infection is caused by an organism for which porphyrin, a porphyrin analog or a porphyrin-like molecule including a porphyrin derivative is an auxotrophic requirement. Particularly preferred target organisms include those for which hemoglobin and its precursors as well as heme are auxotrophic requirements.

The porphyrin macrocycle resembles an expanded benzene ring system where the planar porphyrin macrocycle ring or part thereof is a highly conjugated system with a number of resonating forms, which allows it to take part in diverse biological processes. In the center of a metalloporphyrin macrocycle lies a central cavity which is available for chelation to metals (Smith, Porphyrins and Metalloporphyrins, Elsevier Scientific Publishing Company, Amsterdam, 1976). However, the present invention contemplates both porphyrins with and without a chelated metal. A non-metalloporphyrin is also referred to herein as a free-based porphyrin.

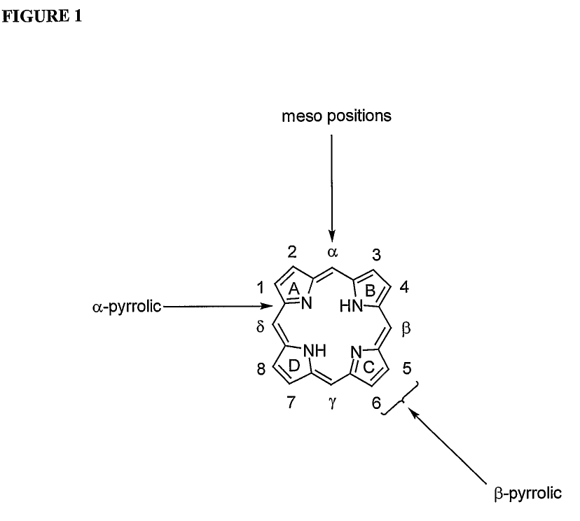

Porphyrins may have a variety of substituents in the β-positions of the pyrrole rings (positions 1-8 in FIG. 1). The meso positions of the porphyrin rings (α, β, γ and δ, as set out in FIG. 1) are generally not substituted except for synthetic compounds such as tetraphenylporphyrin (Cannon, 1993, supra). However, porphyrin-like molecules comprising substitutions in the meso positions are within the scope of the present invention.

Porphyrins have demonstrated important antibacterial activity against a range of bacterial strains and exert their antibacterial properties by interfering with the bacterial cell's ability to absorb and metabolize essential requirements such as iron. It is proposed that the binding site of heme in P. gingivalis is the HA2 domain and heme recognition by HA2 may be solely porphyrin mediated. The HA2 containing proteins are proposed herein to be able to recognize the porphyrin in either a flat planar structure (iron bound) or, a slightly “buckled” structure (non-iron complexes). However. metal-based transport systems such as the HasA system is also contemplated by the present invention.

In one embodiment, the TMAs of the present invention are adapted to treat an infection in an animal including human subject, wherein the infection is caused by P. gingivalis for which porphyrin, a porphyrin analog or a porphyrin-like molecule is an auxotrophic requirement.

Reference herein to “Porphyromonas gingivalis” or its abbreviation “P. gingivalis” includes reference to all mutants, derivatives and variants of this organism as well as serological sub-types. The present invention further extends to microorganisms related to P. gingivalis at the metabolic, structural, biochemical, immunological and/or disease causing levels. Examples of related microorganisms include but are not limited to Salmonella spp., Serratia spp., Yersinia spp., Klebsiella spp., Vibrio spp., Pseudomas spp., E. coli and Haemophilus spp.

Porphyromonas gingivalis has a requirement for heme as a growth factor and unlike most organisms, iron is not required as an essential recognition factor. Instead the HA2 receptor recognizes the porphyrin macrocycle. Porphyromonas gingivalis is unable to biosynthesize the porphyrin macrocycle for its metabolic requirements as it lacks a number of enzymes involved in porphyrin synthesis as demonstrated by perusal of the genomic sequence. Therefore, the presence of exogenous heme or other porphyrins is essential for the growth of P. gingivalis.

Porphyromonas gingivalis is a major pathogen of chronic destructive adult periodontitis and its success as a pathogen in the complex microbial community of destructive periodontitis may be attributed to the capacity of the cysteine proteases to promote bleeding.

Heme function in P. gingivalis requires the cell surface protein Kgp for hemolysis, hemoglobin proteolysis and efficient heme capture. Porphyromonas gingivalis contains lysine-specific Kgp and arginine-specific RgpA cysteine proteinases, known as gingipains, which are multidomain proteins that contain a cysteine protease domain and additional C-terminal domains HA1 to HA4, which comprise the hemagglutinin domain.

It is proposed that the binding site of hemoglobin in P. gingivalis is the HA2 domain and is part of three HA2-containing proteins: gingipains Kgp, RgpA and the putative hemagglutinin protein HagA. As the HA2 domain is conserved in gingipains, this allows the utilization of this feature for targeted inhibition. As indicated above, it is proposed that non-metal porphyrins (buckled morphology) and metalloporphyrins (planar morphology) may be used by the HA2 system.

The HasA receptor from the organism Serratia marcescens illustrates a typical hemophore for heme capture by most organisms. The heme/HasA binding interaction is fundamentally different to the heme/HA2 binding interaction. The crystal structure of the heme/HasA complex indicated that heme is located at the interface between two parts of the molecule, based on iron coordination to His32 and Tyr75.

In contrast, binding of heme to HA2 is not through iron coordination. Binding studies using recombinant proteins indicated binding of heme to the HA2 domain by an interaction with the propionate groups of the heme moiety. It is also believed that there is some lateral recognition of porphyrin wall although there are no strict requirements on the vinyl aspect of the porphyrin for recognition. This allows the derivitization of the porphyrin macrocycle at certain positions to incorporate harmful molecules to selectively inhibit the targeted organism, P. gingivalis.

It is unnecessary for iron to be coordinated to the porphyrin for its uptake into the cell to support growth of P. gingivalis. Indeed, it is probably advantageous to avoid using metalloporphyrins in the context of developing a selective drug against P. gingivalis because in this way inhibition of other organisms by a HasA-type binding interaction is prevented, making for a much more selective drug. Previous work has also shown that the propionic acid groups at positions 6 and 7 of PPIX are important for its uptake by P. gingivalis. Notwithstanding, the present invention contemplates both free-based (non-metallo) porphyrins as well as metalloporphyrins.

When P. gingivalis cells are starved of porphyrin, they are unable to proliferate even under iron-replete environments. However, when the cells are treated with porphyrin soon after the stationary phase, the heme-starved cultures recover. As both metallated (heme) and free-based porphyrins (PPIX and DPIX) produce similar recovery profiles, it provides evidence that the cells capture and respond to porphyrins in an iron independent manner.

As used herein the terms “porphyrin”, “porphyrin-like molecule” and “porphyrin analog” should be understood to encompass any molecule which comprises a porphyrin macrocycle ring as shown in FIG. 1.

In a preferred embodiment of the present invention, the targeting moiety of the TMA is a porphyrin analog. In other words, the porphyrin analog is the vehicle to deliver any molecule or agent linked, conjugated or associated to it to an organism requiring porphyrin.

As used herein, the term “porphyrin analog” should be understood to encompass a porphyrin-like molecule comprising one or more substituents at any one the β-positions of the pyrrole rings (positions 1-8 in FIG. 1) or the meso positions of the porphyrin rings (α, β, γ and δ, as set out in FIG. 1). Accordingly, the term “porphyrin analog” specifically includes, but is in no way limited to the porphyrin analogs set out below:

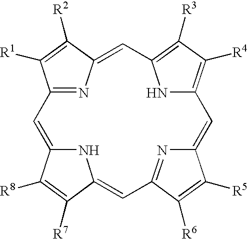

wherein:

| | DPIX | PPIV | PPVI | PPIX | PPXII | PPXIII | PPXIV |
|---|---|---|---|---|---|---|---|
| | 5 | 6 | 7 | 3 | 8 | 9 | 10 |
| R¹ | —CH₃ | —CHCH₂ | —CH₃ | —CH₃ | —CH₃ | —CHCH₂ | —CH₃ |
| R² | —H | —CH₃ | —CHCH₂ | —CHCH₂ | —CHCH₂ | —CH₃ | —CHCH₂ |
| R³ | —CH₃ | —CH₃ | —CHCH₂ | —CH₃ | —CHCH₂ | —CH₃ | —CH₃ |
| R⁴ | —H | —CHCH₂ | —CH₃ | —CHCH₂ | —CH₃ | —CHCH₂ | —CHCH₂ |
| R⁵ | —CH₃ | —(CH₂)₂CO₂H | —CH₃ | —CH₃ | —(CH₂)₂CO₂H | —CH₃ | —(CH₂)₂CO₂H |
| R⁶ | —(CH₂)₂CO₂H | —CH₃ | —(CH₂)₂CO₂H | —(CH₂)₂CO₂H | —CH₃ | —(CH₂)₂CO₂H | —CH₃ |
| R⁷ | —(CH₂)₂CO₂H | —CH₃ | —CH₃ | —(CH₂)₂CO₂H | —CH₃ | —(CH₂)₂CO₂H | —(CH₂)₂CO₂H |
| R⁸ | —CH₃ | —(CH₂)₂CO₂H | —(CH₂)₂CO₂H | —CH₃ | —(CH₂)₂CO₂H | —CH₃ | —CH₃ |

It has been indicated that substituents in the β-positions of the pyrrole rings (1-8) are important in defining the therapeutic properties of the porphyrin and generally the meso positions of the porphyrin rings (α, β, γ and δ) are unsubstituted. The vinyl groups at positions 2 and 4 of PPIX as side chains cause the porphyrin molecule to be unstable. If the vinyl groups of PPIX are replaced with groups that do not provide conjugation to the molecule, such as hydrogens to give DPIX, the stability of the molecule is greatly enhanced.

However, is should be understood that the term “porphyrin analog” as used herein encompasses porphyrin analogs comprising a substitution at one or more of the meso positions of the porphyrin macrocycle. Examples of substitutes include monosulfonic acid and disulfonic acid derivatives at all available positions on the porphyrin periphery (e.g. 2-, 4-, α, β and δ meso positions). 2-sulfonic acid and 4-sulfonic acid derivatives of any compound exemplify herein (including compounds 20 and 21) are particularly preferred.

Binding studies using recombinant proteins indicated binding of heme to the HA2 domain of P. gingivalis by an interaction with the propionic acid groups of the heme moiety. Therefore, preferred porphyrin analogs are those which retain at least one of the propionic acid groups for recognition by HA2. Previous work has also investigated the role of functionality at positions 1-4 and modifications of the propionic side chains at positions 6 and 7. It has been shown that the only tolerated functional group recognized by the HA2 domain of P. gingivalis at positions 1 and 3 is the methyl group and the list of tolerated functional groups at positions 2 and 4 include hydrogen as in deuteroporphyrins, methyl groups, vinyl groups as in protoporphyrins, sulfonic acid groups and the deuteroporphyrin bis-ethylene glycol group (Table 2).

TABLE 2
Tolerated functional groups at the vinyl
face of the porphyrin for HA2 binding.

| Substituents | C1 | C2 | C3 | C4 |
|---|---|---|---|---|
| —H | — | YES | — | YES |
| —CH₃ | YES | YES | YES | YES |
| —CHCH₂ | NO | YES | NO | YES |
| —SO₃H | — | YES | — | YES |
| —CHOHCH₂OH | — | YES | — | YES |

It has also been shown that changes to the propionic acid side chain groups have been shown to inhibit normal growth of P. gingivalis. It was therefore proposed that sulfonic acid groups be added at positions 2 and 4 of the porphyrin as a change in the functionality at those positions to sulfonic acid groups would not greatly alter the binding to the proposed HA2 domain.

In a preferred embodiment therefore, the targeting moiety comprises a porphyrin analog comprising at least one propionic side chain, and in a more preferred embodiment the porphyrin analog comprises at least two propionic side chains.

In one particular embodiment, the present invention provides a TMA comprising the general Formula (I):

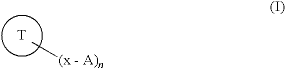

wherein T is a targeting moiety comprising a porphyrin analog, x is a chemical linkage or linker group, A is agent required to be targeted to the organism and n is an integer between 1 and 4, wherein (x−A)₁, (x−A)₂, (x−A) and (x−A)₄, may be the same or different and wherein each is independently linked to the targeting moiety.

As indicated above, x may or may not be needed depending on the means of linking, conjugating or associating A to T.

In a particularly preferred embodiment, the porphyrin analog comprises DPIX.

In one embodiment the targeted agent (A) may be any agent which has a microbiocidal or mircobiostatic effect against one or more target organisms. For example, targeted agents include, but are in no way limited to anti-bacterials, antifungals and anti-parasitic compounds. These may be naturally occurring or chemically delivered.

Exemplary agents of the present invention include but are not limited to: Aminoglycosides (including gentamicin, neomycin and streptomycin), Beta-lactams, Penicillins (including Ampicillin, Amoxicillin, Co-amoxiclav and Flucloxacillin), Cephalosporins (including Cefalexin, Cefaclor and Cefuroxime), Chloramphenicol, Cycloserines, Ionophores, Glycopeptides, Lincosamides, Macrolides (including Erythromycin and Clarithromycin), Monobactams, Polypeptide antibiotics, Nitroimidazoles (including Metronidazole, Nimorazole and Timidazole), Quinolones (including Ciprofloxacin), Stretogramins, Sulfonamides, Tetracyclines (including Tetracycline, Doxycycline, Oxytetracycline), bambermycin, carbadox, novobiocin, spectinomycin, Clindamycin, Isoniazid, Rifampicin, Trimethoprim (Monoprim) and Vancomycin.

The targeted agent may also include the pro-form of an agent, that is a compound which only attains activity or full activity once the compound is modified and/or metabolized in a biological system or organism.

Metronidazole is especially effective against anaerobic infections, such as P. gingivalis infections because in anaerobic conditions, the metronidazole molecule changes so as to inhibit the DNA repair enzymes that would normally repair cells in anaerobic conditions leading to death of anaerobic bacteria, but having no effect on aerobic tissues. The primary action of metronidazole is a rapid inhibition of DNA replication (Sigeti et al., J Infect Dis. 148: 1083-1089, 1983) by causing DNA strand breakages at concentrations easily attainable during routine drug administration, but which only occurs after ferredoxin-mediated reduction of the nitro group (Edwards et al., Janssen Research Foundation Series 2:673-676, 1980). The nitro group of metronidazole once transported into the body gets reduced in situ in the body via oxime and hydroxylamine to an amine. The oxime and hydroxylamine intermediates are encompassed by the present invention. Under anaerobic conditions, the nitroimidazoles are metabolically reduced to intermediates which are toxic (Olive, Brit. J. Cancer 40: 94-104, 1979). Metronidazole is used in radiotherapy for cancer as the inhibition of DNA repair enzymes can sensitize anaerobic tissues to radiation. Metronidazole is also an effective antibiotic against certain protozoal infections, especially Giardia spp. Metronidazole is active only when it is in the unstable amino form, hence is used as a prodrug.

It has been shown that there is excellent activity of metronidazole against P. gingivalis (Poulet et al., J. Clin. Periodontol. 26: 261-263, 1999; Mitchell, J. Clin. Periodontol. 11: 145-158, 1984) Also it is an antibiotic to which susceptible anaerobes have yet to develop clinical resistance, therefore it is the preferred drug against anaerobic infections (Ghayoumi, Journal of the Western Society of Periodontology/Periodontal abstracts 49: 37-40, 2001). One major disadvantage of using metronidazole is that metronidazole has a very broad spectrum of activity and therefore is not selective to P. gingivalis.

Therefore, in one particularly preferred embodiment, the targeted agent is metronidazole.

Accordingly, the present invention contemplates a targeted molecular agent (TMA) comprising metronidazole or a derivative thereof comprising an NO₂ group in a reduced form, said metronidazole linked via a chemical linkage bond to a porphyrin molecule or an analog or derivative thereof.

In one embodiment, the porphyrin is linked to the metronidazole by a propionic acid group.

In addition, the porphyrin may be a non-metalloporphyrin or a metalloporphyrin. A “reduced form” of NO₂ includes an oxime or a hydroxylamine. The chemical linkage group may be inter alia an ester or an amide bond.

The one or more targeted agents may be bound, chemically linked or conjugated or otherwise associated with the targeting moiety using any means that is evident to one of skill in the art.

In one preferred embodiment, the targeting moiety is a porphyrin, porphyrin analog or porphyrin-like molecule which comprises at least one, and more preferably at least two propionic side chains, and at least one of these side chains is used to form an ester linkage between the carboxyl group of the propionic group and a targeted agent comprising a hydroxyl group.

In one specific preferred embodiment, the present invention contemplates a TMA comprising DPIX as a targeting moiety with metronidazole-substituted adducts comprising an ester linkage through the propionic acid side chains at positions 6 and 7 of DPIX and the hydroxyl group of metronidazole. The hydroxyl group of metronidazole is particularly convenient for attachment to the propionic side chains at positions 6 and 7 by an ester linkage because the only difference between timidazole and metronidazole is that timidazole has a sulfone moiety instead of a hydroxyl moiety but has also been proven active against anaerobes. This indicates that the hydroxyl group on metronidazole may not be essential for activity against anaerobes.

Accordingly, the present invention further contemplates, a TMA comprising the chemical structure of any one of compounds 19, 20 or 21, or isomers thereof:

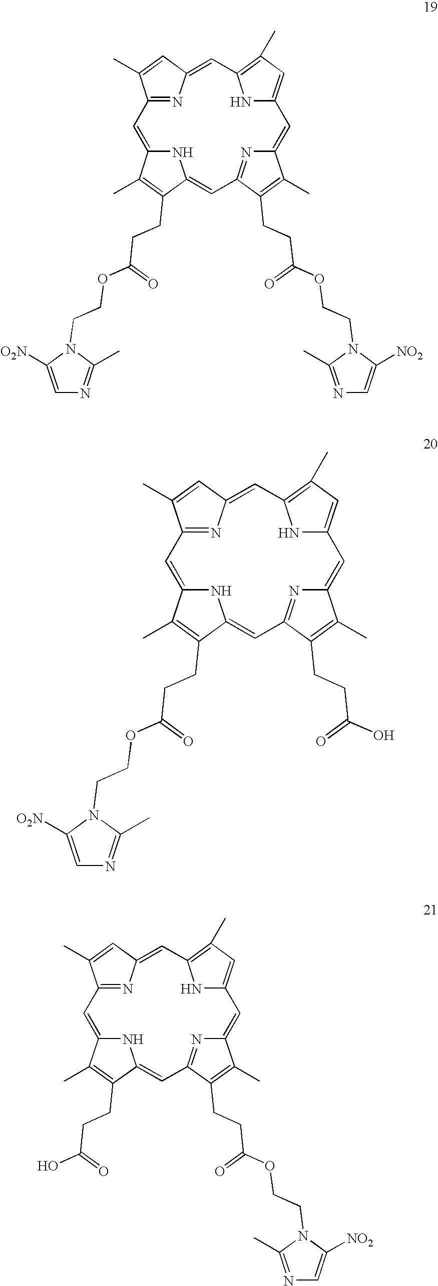

In accordance with the present invention, it is proposed that P. gingivalis only recognizes one side of the porphyrin macrocycle and preferentially binds that side. Therefore, TMAs which are mono-adducts, comprising an targeted agent only bound to one of the propionic groups, leaving one propionic acid group on the porphyrin macrocycle intact may assist recognition, binding and uptake of the TMA into P. gingivalis. Accordingly, in one preferred embodiment, the TMAs of the present invention comprise at least one “free” propionic group, for example, compounds 20 and 21.

The present invention also contemplates a TMA comprising an amide spacer incorporating amino acids such as arginine (Arg/R) or lysine (Lys/K), as a linker between the targeted agent and the propanoic acid side chain or chains of the porphyrin molecule to allow for selective hydrolysis of the targeted agent once it is bound at the cell surface or outer membrane of the target pathogen.

The formation of a peptide bond between an amino acid and a carboxylic acid is an example of a condensation reaction whereby the molecules may be joined together with the accompanying removal of a molecule of water. This should be particularly useful as the gingipains are arginine and lysine specific and is a rich source of cysteine proteases, which account for a high proportion of the proteolytic activity of dental plaque. This would mean increased selectivity to P. gingivalis as other organisms do not have gingipains and as a result the amide spacer cannot be easily cleaved by other organisms but can be selectively cleaved by P. gingivalis.

For example, with respect to metronidazole, several approaches are available. One involves substitution of a propionic acid group (see Scheme A) another (Scheme B) requires prior derivatisation of the macrocycle backbone. Linkage through a porphyrin meso-position by first placing an amino group there, or by reducing the nitro group of metronidazole (which happens in vivo, metronidazole is a pro-drug) and then forming an amide bond using it, are also possible.

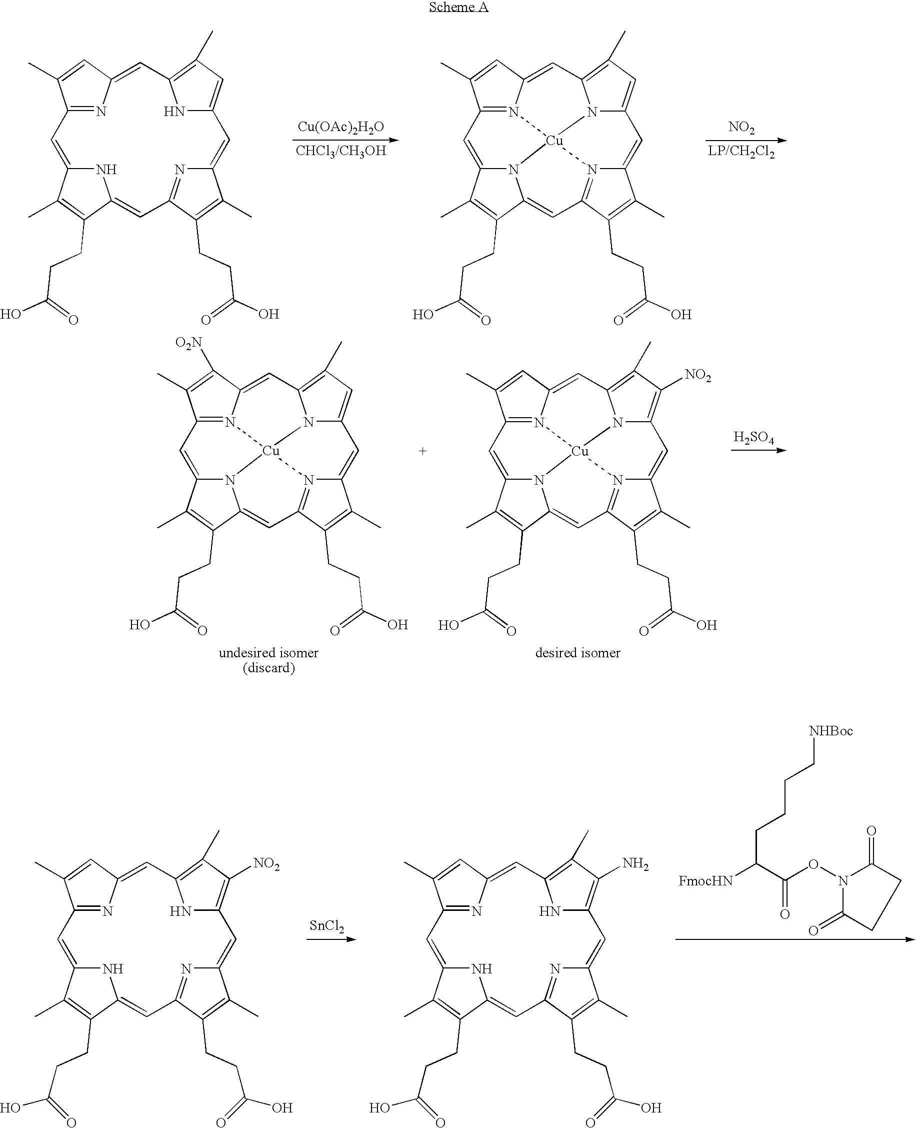

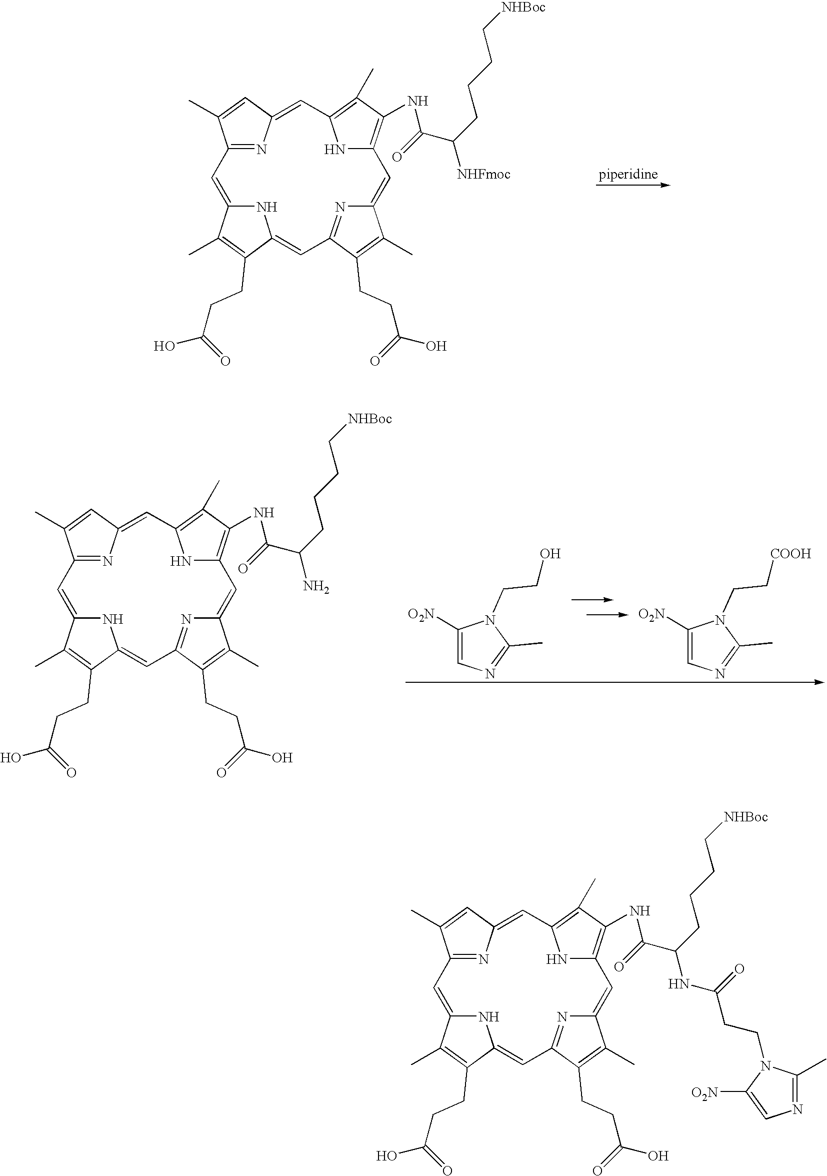

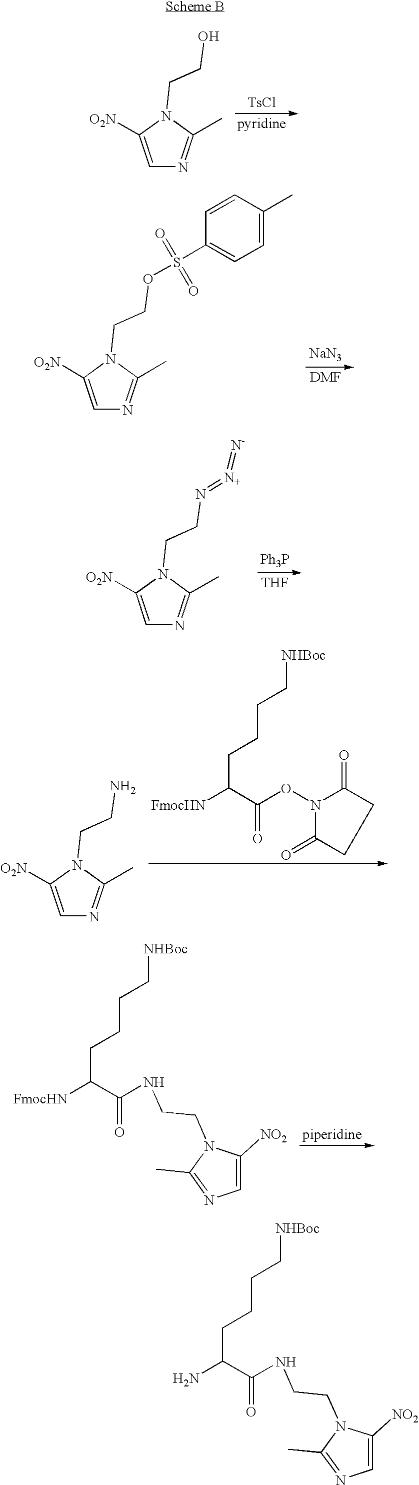

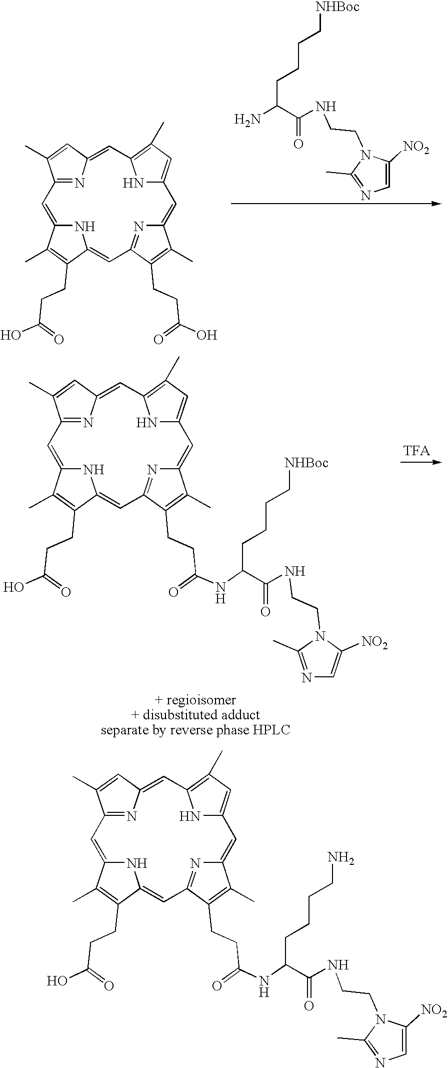

It is also envisaged that the synthetic chemistry shown in Scheme A can be directed towards the generation of an urea linkage between amino groups on porphyrin derivatives and an amino group of a metronidazole derivative. It is also envisaged that Scheme B can be modified to generate a carbamate ester linkage can be made between amino groups on porphyrin derivatives and the hydroxyl of the parent compound, metronidazole.

The present invention further extends to TMAs comprising linkage of a targeted agent to the porphyrin, porphyrin analog or porphyrin-like molecule including a vinyl group at position 2 and/or 4 of the porphyrin macrocycle. Furthermore, the present invention contemplates the addition of a sulfonic acid group at one of the vinyl groups (positions 2 and/or 4). In this context the “therapeutic” nature of the agent is in respect of its ability to kill or inhibit organisms such as those causing acute or chronic infection.

In another embodiment, the present invention further extends to TMAs comprising multiple targeted agents bound, chemically linked or otherwise associated with a targeting moiety. For example, multiple targeted agents may be linked to the porphyrin, porphyrin analog or porphyrin-like molecule via one or more or of the vinyl groups and/or propionic acid groups. Accordingly, a single porphyrin, porphyrin analog or porphyrin-like molecule may comprise one, two, three or four associated targeted agents.

Exemplary compounds TMAs comprising multiple targeted agents (for example metronidazole) are shown as compounds 39, 40, 41 and 42 and isomers thereof, where substitution of the targeted agent, such as metronidazole occurs through one of the vinyl positions and one of the propionic side chains.

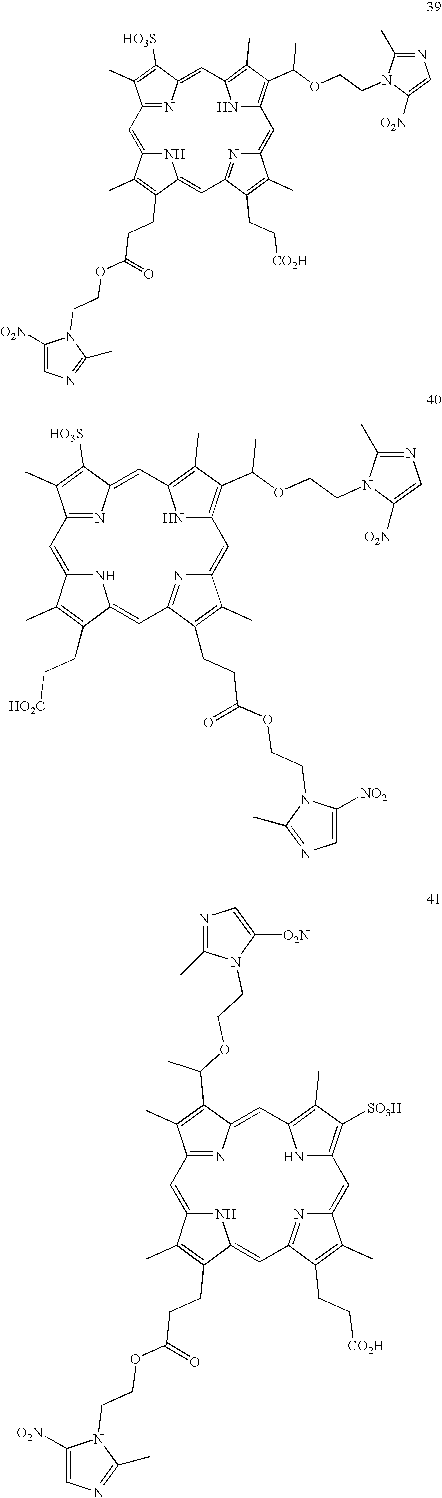

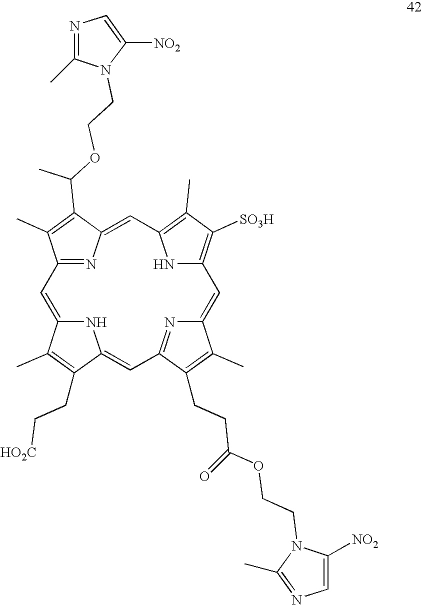

The present invention further extends to tri-substituted-adducts, such as compounds 43 and 44, or issues thereof where only one propanoic side chain is left intact for binding by the target organism.

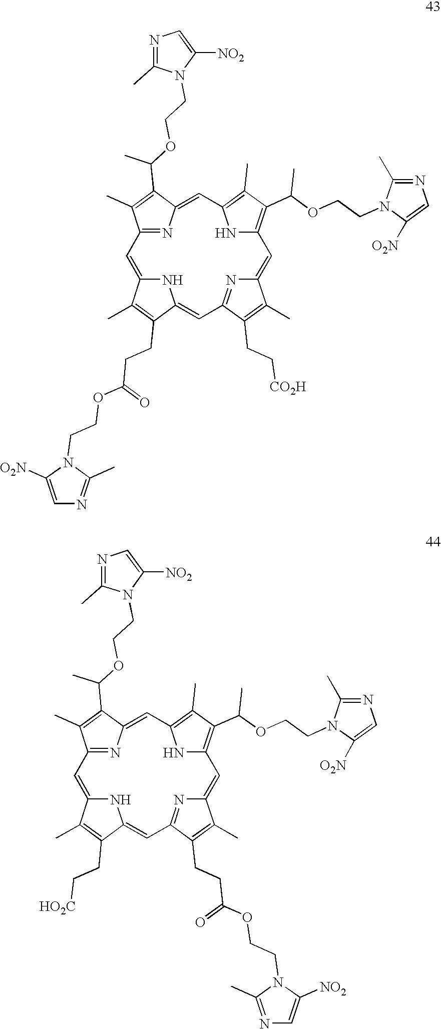

Although exemplified using metronidazole as the targeted agent, the present invention extends to TMAs comprising any suitable targeted agent which may be bound to the targeting moiety.

The TMAs of the present invention may be synthesized using any methods that would be apparent to those of skill in the art. An exemplary synthesis strategy for the production of a TMA comprising a DPIX targeting moiety conjugated to metronidazole is presented below and in the Examples. However, it should be understood that the present invention is in no way limited to the exemplified TMA or the exemplified methods of synthesis.

Deuteroporphyrin IX (DPIX) 5 was chosen as the precursor for the production of the DPIX targeting moiety. Deuterohemin 23 was prepared from protohemin (Scheme 1) following the method of Smith J. Porphyrins Phthalocyanines 1999, 4, 319-324.

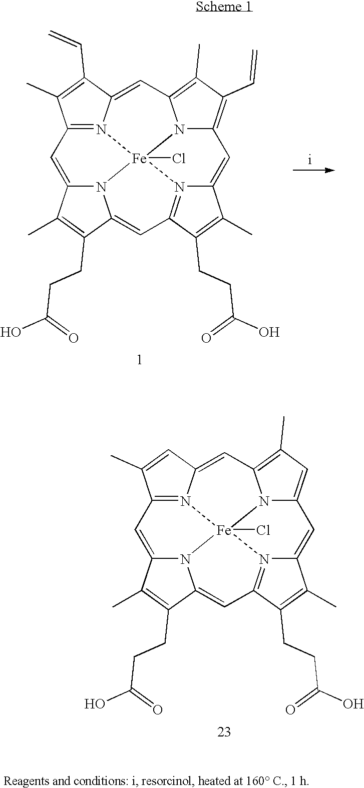

This was done by heating protohemin in a resorcinol melt, the Schumm protiodevinylation reaction, where the vinyl groups at positions 2 and 4 are replaced by hydrogens in this reaction. This mechanism involves electrophilic substitution reactions on the phenolic reagent to yield crude deuterohemin 23. This reaction was then washed with diethyl ether until the washings were colourless to remove any residual unreacted resorcinol, impurities and any resorcinol polymers that had formed to yield pure deuterohemin 23 in 98% yield. Confirmation of the structure was evidenced by MALDI-TOF mass spectroscopy, which gave a parent ion peak at 564.2 [(M)⁺ requires 564.4].

Another method to synthesize deuterohemin 23 was also performed. This followed the method of Adler et al. (Bioinorg Chem. 1977, 7, 187-188), whereby hot propionic acid was added after the melt had cooled to approximately 140° C. and the resulting solution was poured into water. The solution was then neutralized with sodium hydroxide and heated for a further 1 h after which the solution was cooled and the fine precipitate was allowed to aggregate into bigger particles. The solution was vacuum filtered with a large sintered funnel as the filtration was slow and the filtrate was filtered until clear. The precipitate was air-dried, dissolved with methanol, the solution was filtered again and the residue was dried in vacuo. Water was added to the crude deuterohemin 23 with heating and the solution was filtered. The water was removed to yield deuterohemin 23 in 76% yield. Confirmation of the structure was evidenced by MALDI-TOF mass spectroscopy, which gave a parent ion peak at 564.2 [(M)⁺ requires 564.4]. The next step was to prepare DPIX 5 from deuterohemin 23 through demetallation and this employed a variety of methods.

Initially, DPIX 5 was prepared from deuterohemin 23 (Scheme 2) following the iron powder method of Smith via DPIX DME 24.

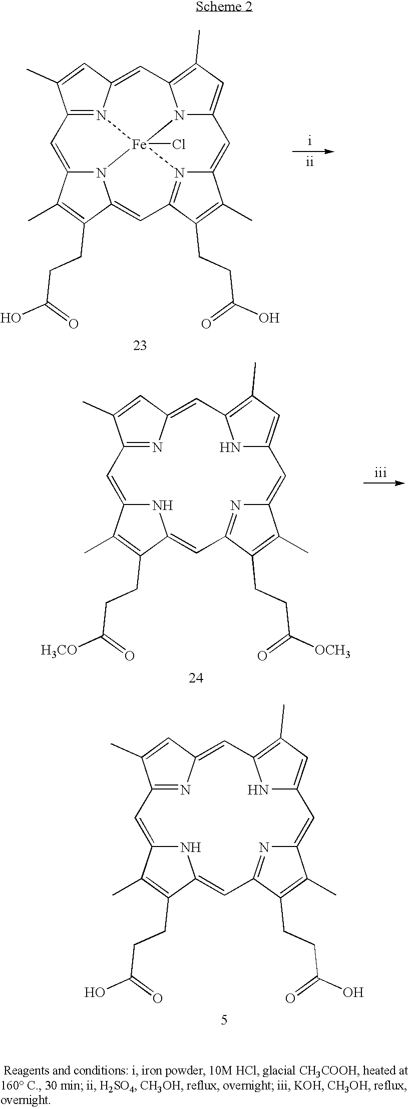

The first step involved the reduction of Fe(III) to Fe(II) with Fe(0) to demetallate deuterohemin 23. As the Fe(III) is reduced to Fe(II), its ionic radius increases and as a result it becomes larger and does not fit as well in the core of the porphyrin. Also, the strong hydrochloric acid protonates the core of the porphyrin to prevent reinsertion of Fe(II) to the porphyrin.

The second step involved a simple esterification of DPIX 5 in methanol and concentrated sulfuric acid to yield crude DPIX DME 24. Confirmation of the structure was proven by MALDI-TOF mass spectroscopy which gave a parent ion peak at 539.3 [(M+H)⁺ requires 539.7]. Chromatography of the crude material through a neutral alumina column with an eluent of 5% methanol/chloroform afforded pure DPIX DME 24 in 61% yield, with an identical ¹H NMR spectrum to that quoted in the literature.

The third step involved hydrolysis of DPIX DME 24 to the corresponding acid 5. The hydrolysis proceeded with acceptable purity, albeit in low yield. The yields obtained for this reaction were in the range of 35-40%. This was inconsistent with the literature precedent of 65% for this compound. Although yields were low, the product DPIX 5 had an identical ¹H NMR spectrum to that quoted in the literature. DPIX 5 was also prepared directly from deuterohemin 23 (Scheme 3).

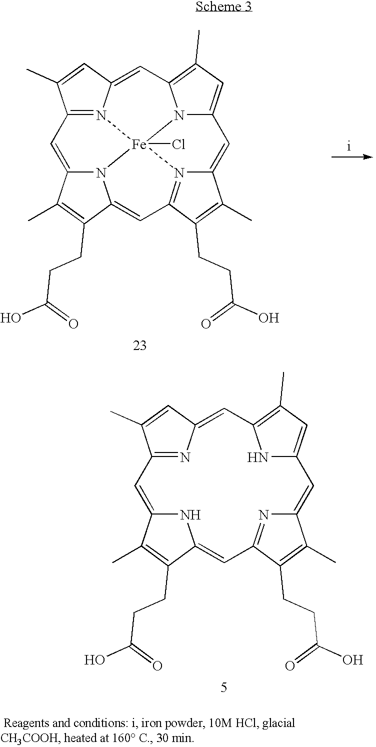

Initially the product from this reaction was produced at a yield of 55-60%. However, the literature method for this reaction is not easily reproducible. When the aqueous layer was extracted with dichloromethane instead of ethyl acetate, both ¹H NMR and MALDI mass spectroscopy showed that very pure product was obtained, albeit in low yield of 12%. The low yield observed led to the conjecture that there were problems present in the work-up of the reaction and it was thought that a significant proportion of the product was being lost due to insufficient extraction from the aqueous layer. However, repeated extractions of the aqueous layer with dichloromethane did not overcome this problem and the yields remained at 12%. Ethyl acetate was found to be the best solvent for use in the work-up extractions as it prevented a lot of porphyrin from crystallizing out of solution. It was concluded that not all of the deuterohemin 23 was demetallated and it was thought that demetallation was unpromising due to the fact that the porphyrin was not very soluble in hydrochloric acid. Therefore another method was investigated whereby the reaction mixture was purged with dry hydrogen chloride gas.

DPIX 5 was then prepared from deuterohemin 23 (Scheme 4) following the ferrous sulfate method of Smith, 1999, supra; 1976, supra.

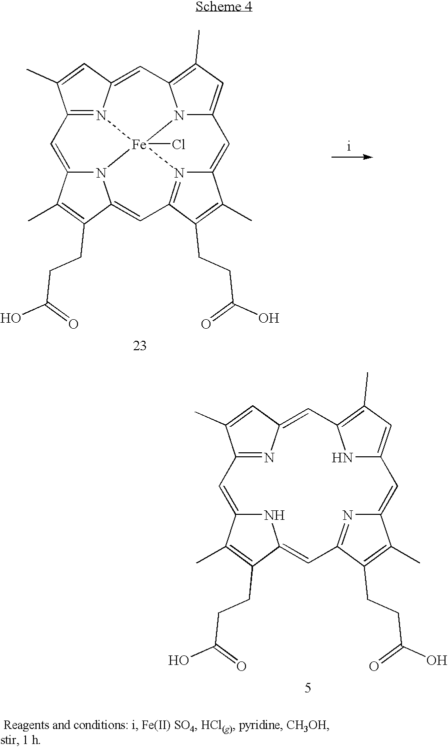

Deuterohemin 23 was dissolved in pyridine and methanol, ferrous sulfate was added and dry hydrogen chloride gas was passed rapidly through the solution. However, this method produced DPIX 5 in only 17% yield, with an identical ¹H NMR spectrum to that quoted in the literature.

A totally different approach to synthesize DPIX 5 was utilized when all the methods to demetallate the porphyrin, deuterohemin 23, were thought to be exhausted. This involved the use of a different precursor, HPIX 25, to form DPIX 5.

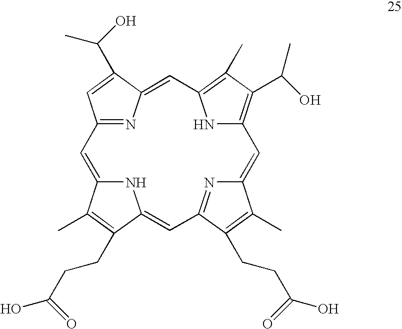

The only other difference between HPIX 25 and protohemin is that the hydroxyethyl groups on HPIX 25 replace the vinyl groups at positions 2 and 4 on protohemin. PPIX was prepared from HPIX 25 (Scheme 5) following the method of Smith, 1999 supra; 1976 supra.

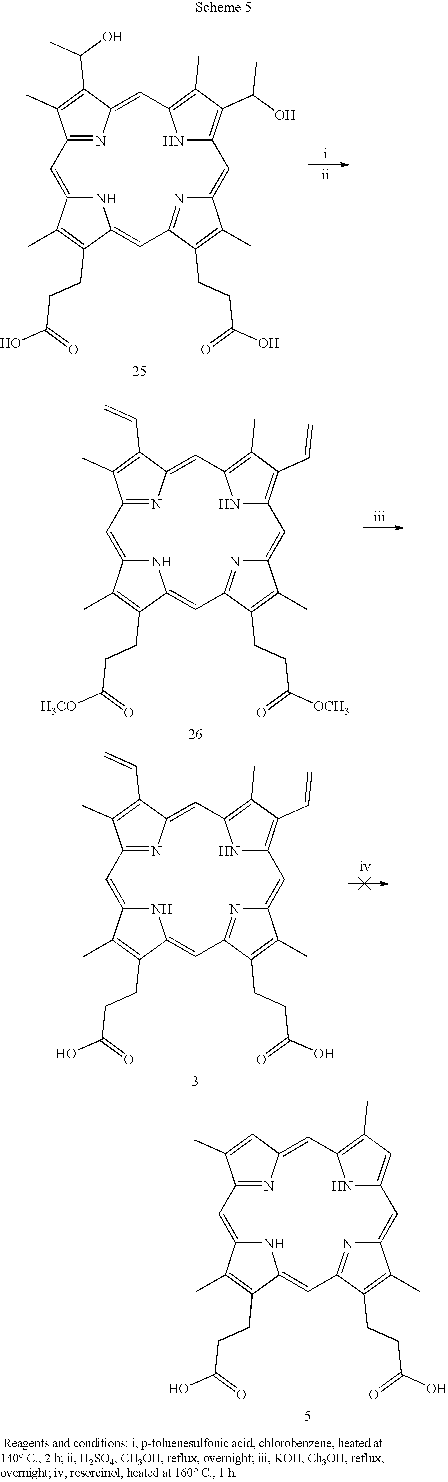

The first step of this procedure involved the acid-catalyzed dehydration of the hydroxyethyl groups at positions 2 and 4 of HPIX 25 to vinyl groups. Chlorobenzene was used instead of dichlorobenzene as its lower boiling point allowed easier removal. Ethyl acetate was used as the extracting solvent as it was found from previous work that PPIX was difficult to crystallize and using ethyl acetate instead of dichloromethane prevented a significant amount of PPIX from crystallizing out of solution.

The second step involved a simple acid-catalyzed esterification in methanol to yield crude PPIX DME 26 as a dark red solid. Confirmation of the structure was proven by MALDI-TOF mass spectroscopy. Chromatography of the crude material through a silica column with an eluent of 2% v/v methanol/chloroform afforded pure PPIX DME 26 in 83% yield, with an identical ¹H NMR spectrum to that quoted in the literature.

The third step involved the hydrolysis of PPIX DME 26 to the corresponding acid PPIX. The hydrolysis proceeded efficiently yielding 77% of PPIX. Analysis of the product by ¹H-NMR and further confirmation of the structure by MALDI-TOF mass spectroscopy allowed the determination of the product to be of sufficient purity to be used without further purification.

The fourth step involved was attempting the devinylation of PPIX to DPIX 5 via the Schumm protiodevinylation reaction. This involved brief heating of PPIX in a resorcinol melt. However, TLC analysis of the product showed that no reaction had occurred. The reaction was repeated with longer heating with resorcinol but TLC analysis still showed that no reaction had occurred. The devinylation was unsuccessful as the Schumm protiodevinylation reaction only works on metallated porphyrins.

It was then suggested that Cu(II) be added to HPIX 25 before dehydration (Scheme 6) and that the reactions would be carried out starting with Cu(II) HPIX 27 instead of HPIX 25. This would allow for the devinylation of Cu(II) PPIX 28 to Cu(II) DPIX and subsequent removal of Cu(II) from Cu(II) DPIX to DPIX 5.

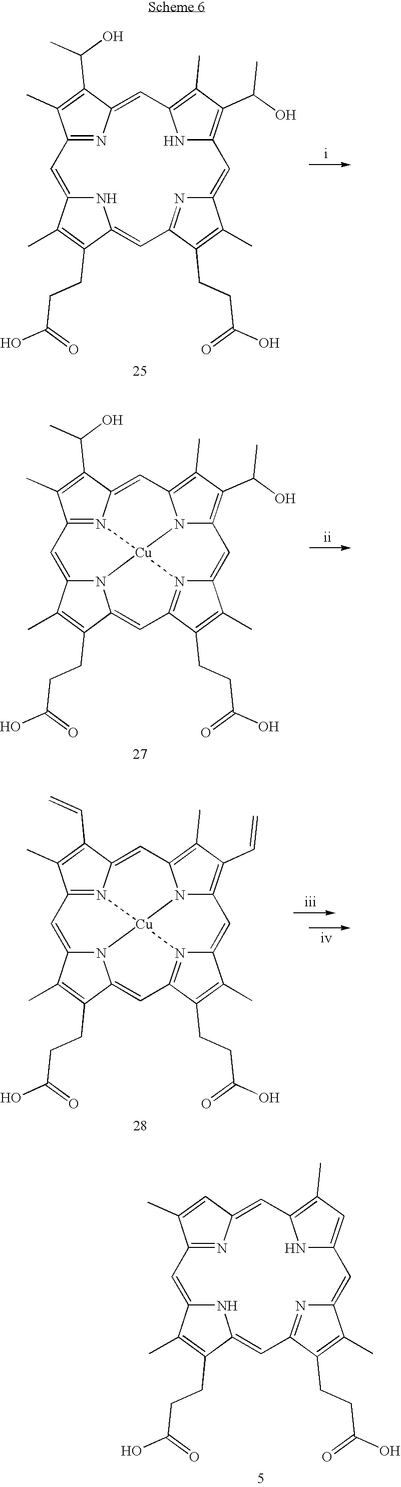

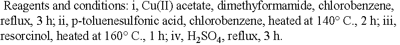

Following the method of Adler, et al. Inorg. Nucl. Chem. 1970, 32, 2443-2445 HPIX 25 and copper(II) acetate were dissolved in dimethylformamide and the solution was heated at reflux for 3 h. Upon cooling, the solvent was evaporated off to yield black crystals. However, both MALDI-TOF and ESI mass spectroscopy suggested that copper(II) HPIX 27 was not obtained and mostly starting material was recovered.

It was determined that both the iron powder and ferrous sulfate methods were flawed in their own respect. The iron powder method was flawed because the porphyrin was found to be poorly soluble in the hydrochloric acid and although the porphyrin was soluble in the acetic acid, acetic acid is too weak to protonate the core of the porphyrin. To solve this problem, formic acid was used in place of hydrochloric acid. Although formic acid is weaker than hydrochloric acid, it is a strong organic acid and is able to readily solvate the porphyrin. Initially, the iron powder method was carried out under atmospheric conditions whereby oxygen and water vapour was present. This meant that the Fe(II) could be oxidized to the Fe(III) state and reinserted into the porphyrin. The reaction was then carried out under oxygen-free conditions by purging the reaction mixture with nitrogen gas and keeping the reaction under nitrogen gas for the whole duration of the reaction. Fe(0) was added at intervals of 5 min and it was observed that the iron porphyrin, deuterohemin 23, had a characteristic brownish colour and the free-based porphyrin, DPIX 5, had a characteristic purple-red colour. By following the reaction by TLC, it was observed that the reaction works best after approximately 15 min. However as the reaction goes for more than 20 min, the reaction reverts back to starting material. The ferrous sulfate method was also flawed because the Fe(II) replaced Fe(0) as the reducing agent and as at least five to ten equivalents of the reducing agent is required to reduce the Fe(III) in the porphyrin, using Fe(II) would result in more Fe(II) being produced which can shift the equilibrium back to the starting material. To solve this problem, Fe(0) was used in place of Fe(II) in the formic acid method.

To optimize conditions for the demetallation of deuterohemin 23, the formic acid method was employed, whereby Fe(0) is used in conjunction with formic acid (Scheme 7).

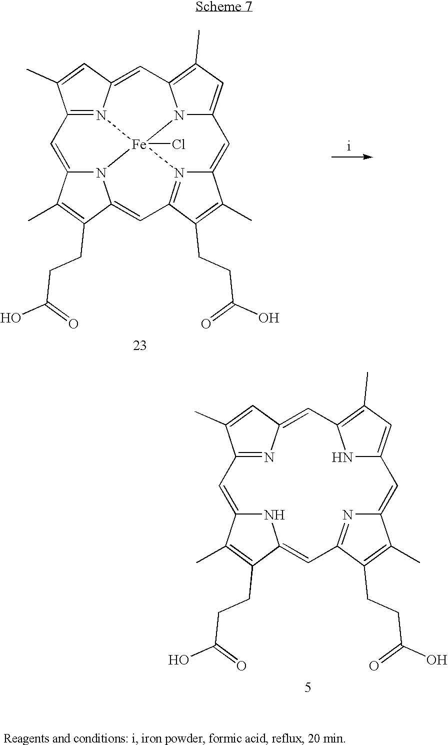

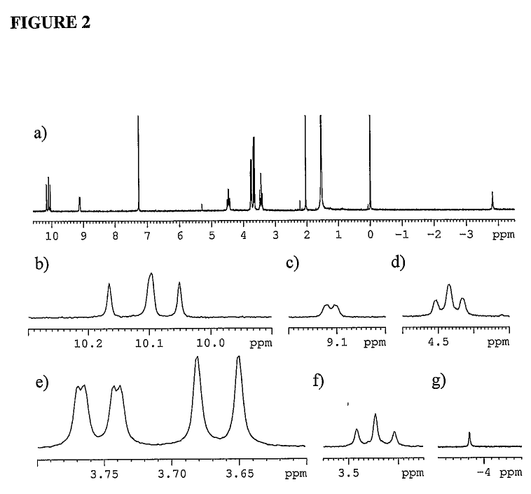

With the conditions optimized, pure DPIX 5 was successfully synthesized in 76% yield as evidenced by the ¹H NMR spectra (FIG. 2), with an identical ¹H NMR spectrum to that quoted in the literature. As Fe is a paramagnetic metal, the attainment of a very clean ¹H NMR spectrum proves successful and complete demetallation. Further confirmation of the structure was proven by high-resolution FTICR mass spectroscopy, which gave a parent ion peak at 511.2320 [C₃₀H₃₀N₄O₄+H⁺] requires 511.2339.

With the synthesis of pure DPIX 5 achieved in good yield, attention was then focused on synthesizing the DPIX metronidazole-substituted adducts 19, 20 and 21 by an ester linkage through the propionic acid side chains at positions 6 and 7 of DPIX 5 and the hydroxyl group of metronidazole. The hydroxyl group of metronidazole was chosen for attachment to the propionic side chains at positions 6 and 7 by an ester linkage because the only difference between timidazole 18 and metronidazole is that timidazole 18 has a sulfone moiety instead of a hydroxyl moiety but has also been proven active against anaerobes. This indicates that the hydroxyl group on metronidazole is not essential for its activity against anaerobes.

As the aim was to synthesize the DPIX mono-metronidazole-substituted adducts 20 and 21, synthesis of the internal anhydride 29 was attempted. This is because it was hoped the reaction of DPIX internal anhydride 29 with metronidazole would allow metronidazole to preferentially attach to only one propionic acid side chain of the porphyrin after ring opening of the anhydride 29 to give the mono-adducts 20 and 21 with no by-products. DPIX 5 was subsequently reacted with acetic anhydride and it was assumed that the internal anhydride 29 was formed (Scheme 8) due to analysis of the reaction by TLC which showed complete transformation of the starting material to a significantly less polar compound.

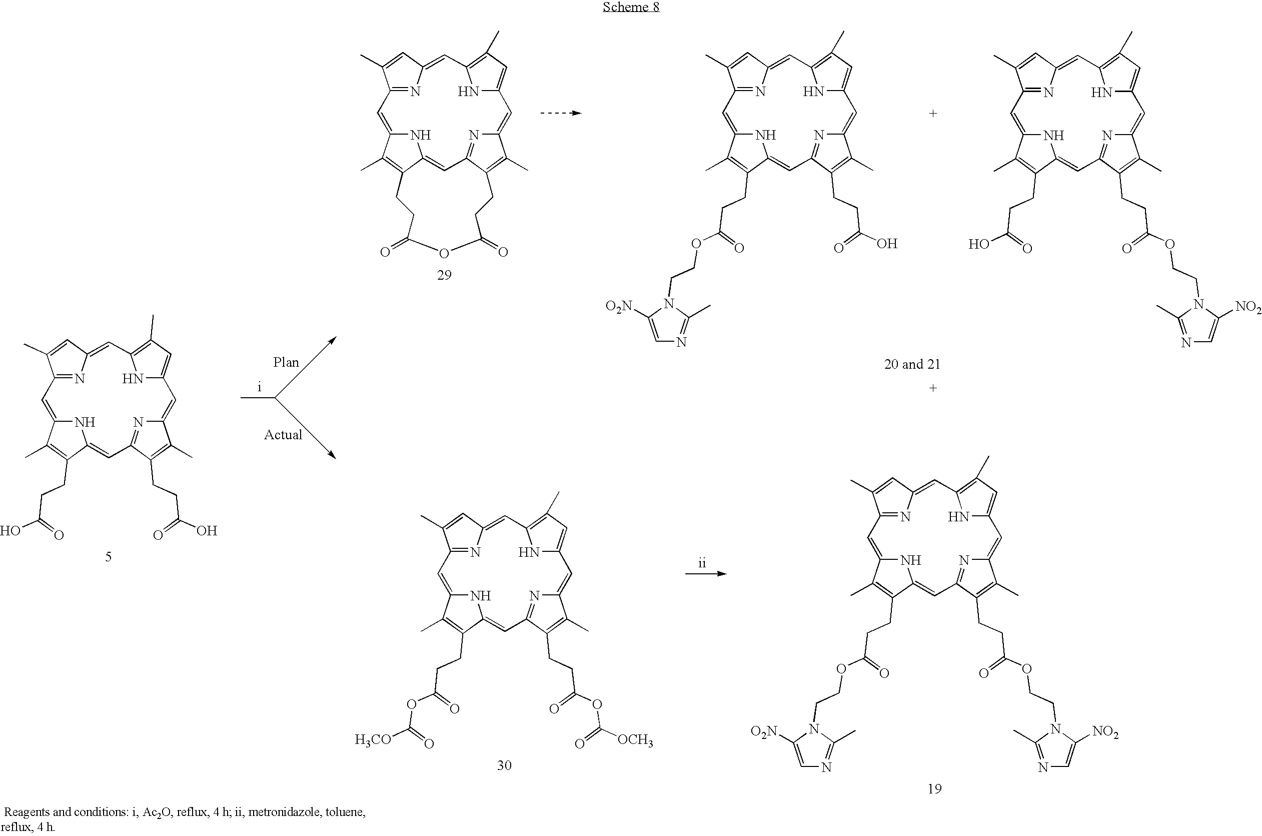

An excess of metronidazole in toluene was then reacted with the intermediate as it was believed that the internal anhydride 29 was the intermediate. However, the reaction proceeded yielding mostly the di-adduct 19 with only trace amounts of a mixture of the mono-adducts 20 and 21. Characterization of the intermediate by MALDI mass spectroscopy showed that the DPIX di-anhydride 30 was formed instead (Scheme 8). This is evidenced by MALDI-TOF mass spectroscopy, which gave a parent ion peak at 595.5 [(M+H)⁺ requires 595.7]. This would explain why mostly compound 19 was formed when the intermediate was reacted with an excess of metronidazole in toluene. Reacting the di-anhydride 30 with less than one equivalent of metronidazole favours the formation of the mono-substituted adducts 20 and 21.

Another approach to synthesizing the DPIX di-adduct 19 and the DPIX mono-adducts 20 and 21 was also investigated. This approach involved activating 5 by forming the di [acid chloride] 31 instead of forming the di-anhydride 30. The idea behind this approach was that the use of a stoichiometric amount of metronidazole would give a stoichiometric mixture of the di-adduct 19, the mixture of the mono-adducts 20 and 21 and the unreacted starting material DPIX 5. Reacting the di [acid chloride] 31 with less than one equivalent of metronidazole would favour the formation of the mono-adducts 20 and 21. DPIX 5 can then be taken through the same procedure to yield more mono-adducts 20 and 21.

DPIX di [acid chloride] 31 was prepared by reacting DPIX 5 with thionyl chloride using dichloromethane as the co-solvent. DPIX di [acid chloride] 31 was then treated with 0.4 equivalents of metronidazole with triethylamine as the base catalyst in toluene to yield the unreacted starting material 5, the di-adduct 19 and the mono-adducts 20 and 21 (Scheme 9).

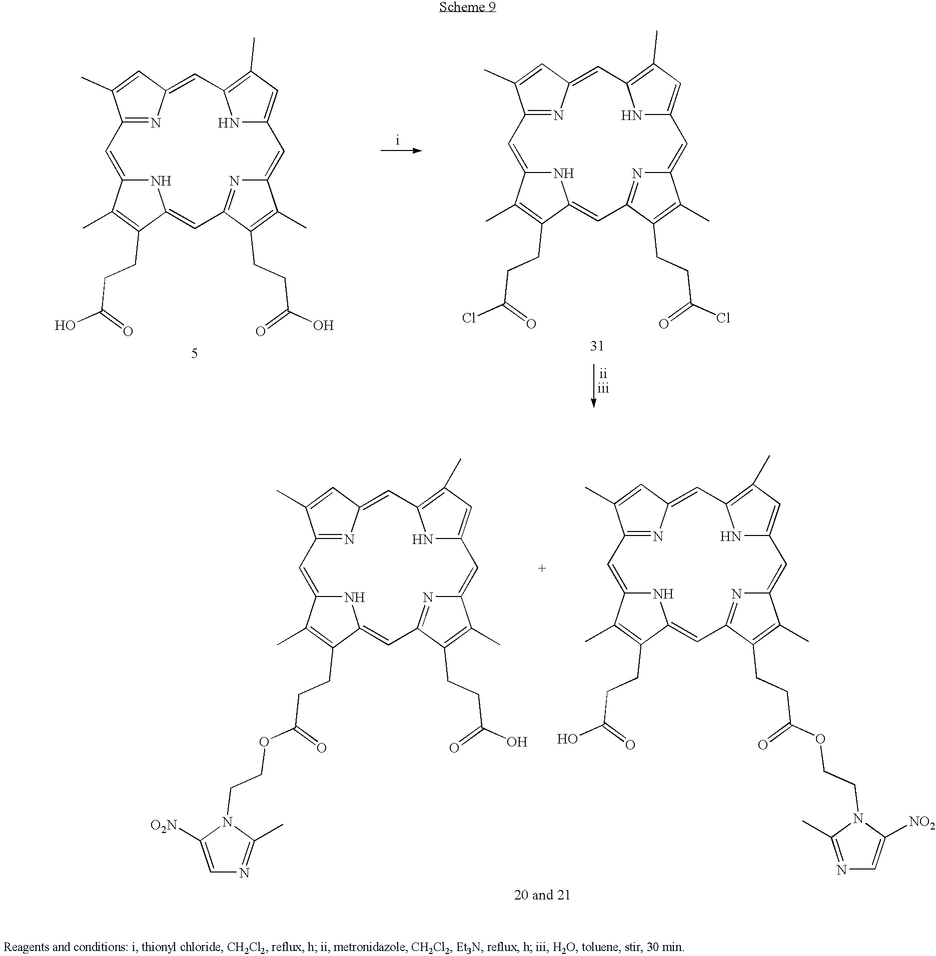

The products were separated on a silica column using a solvent system of CH₂Cl₂:CH₃OH:CH₃NO₂ and initial analysis of the different bands by ESI mass spectroscopy showed 8 extra peaks corresponding to the two mono metronidazole mono methyl ester products, the two mono acid mono methyl ester products, the two mono acid chloride mono methyl ester products, DPIX DME 24 and the DPIX di [acid chloride] 31.

To prevent the formation of the methyl esters, water was added soon after the reaction of metronidazole with the di [acid chloride] 31. This hydrolyzes any unreacted acid chloride back to the free carboxylic acid 5, thereby avoiding formation of the methyl esters. Following that, a mixture of toluene and water was added and the two-phase mixture was stirred for 30 min before evaporating to dryness. Toluene was chosen as the solvent for use in the two-phase system because the mixture of toluene and water forms an azeotrope, which allows for easier removal of the water. This reduced the number of products in the mixture from twelve to four.

The mixture of 5, 19, 20 and 21 were separated by passing through a silica column with a solvent system of CH₂Cl₂:CH₃OH:CH₃NO₂. The initial polarity of the solvent system was 30:1:1. The polarity was increased to 20:1:1 upon which the first band eluted and then to 10:1:1 upon which the second band eluted. The unreacted starting material 5 remained on the baseline of the column as it is the very polar diacid DPIX 5. Initially, the diacid DPIX 5 did not come off the column even with neat methanol. The solvent was then changed to a 1:1 mixture of CH₃OH:NH₃ upon which the DPIX 5 eluted. This is because the ammonia deactivates the silica. DPIX 5 was stripped from the column with a 1:1 mixture of CH₃OH:NH₃ and was recycled to synthesize more mono-adducts 20 and 21. The fractions containing the first band were combined and evaporated to dryness to yield the di-metronidazole-substituted adduct 19.

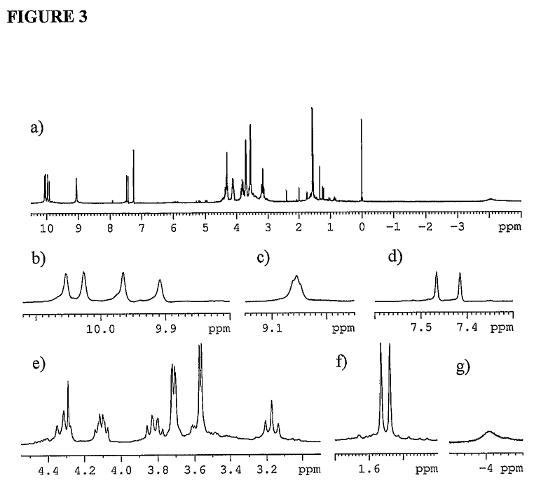

The ¹H NMR spectrum of the di-adduct 19 illustrated in FIG. 3 shows two characteristic singlets at 1.54 and 1.56 ppm due to the methyl group on each of the imidazole rings, as well as two characteristic singlets at 7.42 and 7.47 ppm attributable to the imidazole methine protons. The singlet at 9.04 ppm is characteristic of the β-pyrrolic hydrogens at positions 2 and 4. Four singlets at 9.91, 9.96, 10.03 and 10.05 ppm are characteristic of the four meso protons of the porphyrin macrocycle. Integration of these peaks resulted in a ratio of 6:2:2:1:1:1:1 respectively indicating that the porphyrin was disubstituted.

Also, the infrared spectrum of the di-adduct 19 showed no hydroxyl stretches but showed characteristic NO₂ stretches at 1661 and 1653 cm⁻¹ attributable to the imidazole rings of the di-adduct 19. Further confirmation of the structure was proven by high resolution FTICR mass spectroscopy, which gave a parent ion peak at 817.3349 [C₄₂H₄₄N₁₀O₈+H⁺] requires 817.3416.

The fractions containing the second band were combined and evaporated to dryness to yield the mixture of mono-substituted-metronidazole adducts 20 and 21.

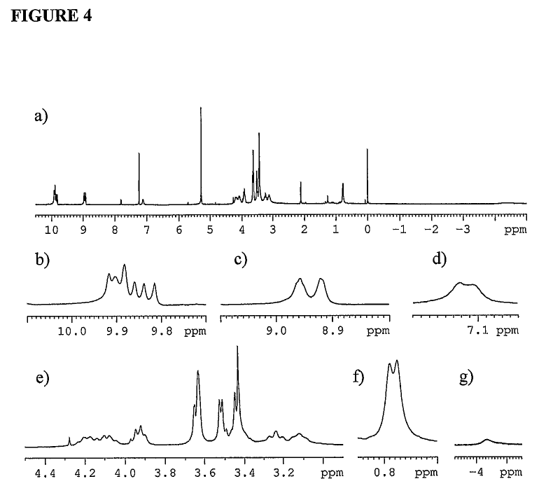

The ¹H NMR spectrum of the mixture of mono-adducts 20 and 21 illustrated in FIG. 4 showed a characteristic singlet slightly split at 0.77-0.79 ppm due to the methyl group on the imidazole ring, as well as a characteristic singlet slightly split at 7.12 ppm attributable to the imidazole methine protons. The singlet slightly split at 8.92-8.96 ppm is characteristic of the β-pyrrolic hydrogens at positions 2 and 4. Four split singlets between 9.81-9.91 ppm are characteristic of the four meso protons of the porphyrin macrocycle. Integration of these peaks resulted in a ratio of 3:1:2:1:1:1:1 respectively indicating that the porphyrin was mono substituted. As can be seen in panel f) of FIG. 4, the two bands are of approximately equal ratio and this suggests that there is an approximately 1:1 mixture of the two mono-substituted adducts 20 and 21.

Also, the infrared spectrum of 20 and 21 showed a hydroxyl stretch at 3136 cm⁻¹ and a NO₂ stretch at 1562 cm⁻¹ attributable to the propionic acid of DPIX 5 and the nitro group on metronidazole of 20 and 21 respectively. Further confirmation of the structure was proven by high resolution FTICR mass spectroscopy, which gave a parent ion peak at 664.2869 [C₃₆H₃₇N₇O₆+H⁺] requires 664.2879.

The activity of a TMA may be assessed using any means that would be evident to one of skill in the art. Typically an activity assay would comprise assays assessing the binding of the TMA to the target and/or assays to assess any microbiostatic or microbiocidal activity on the target organism. In addition, further assays to assess the activity of the TMA on non-target species may also be undertaken.

Exemplary binding assays which may be used to assess the binding of a TMA to a molecular target include ELISA, surface plasmon resonance (Georgiadis et al., J. Am. Chem. Soc. 122: 3166-3173, 2000; Nelson et al., Anal. Chem. 73: 1-7, 2001), electrophoretic mobility shift assays, quartz crystal microbalance assays (Caruso et al., Anal. Chem. 69: 2043-2049, 1997) and the like. However, the target binding capacity of the TMAs of the present invention may be assessed using any convenient method, and the present invention is in no way defined or limited by any one method for assessing the TMAs.

In one specific embodiment of the invention a TMA comprising DPIX conjugated to metronidazole as a mono-substituted adduct, was investigated for the ability to bind proteins comprising the HA2 domain of P. gingivalis.

This was done by establishing the KD₅₀ to which the mono-adducts 20 and 21 binds HA2. Heme was used as a standard because it is known that heme binds very well to HA2 with a binding dissociation constant of ˜15 nM.

The KD₅₀ of the mono-adducts 20 and 21 to HA2 was established using Enzyme-Linked ImmunoSorbent Assay (ELISA), whereby a 96-well polystyrene plate was coated with either the mono-adducts 20 and 21 or hemin in an alkaline coating buffer at pH 9.0. The plates were then washed and blocked for 30 min with a phosphate buffered saline solution containing Tween-20, a strong detergent to prevent any non-specific binding. Dilutions of HA2 in a slightly acidic acetate buffer at pH 5.5 were then titrated down the rows of the 96-well plate and the plate was incubated in an IR Sensor CO₂ incubator at 37° C. for 1.5 h to allow binding of either the mono-adducts 20 and 21 or hemin to HA2. A primary antibody from mouse, anti-gingipain monoclonal antibody 5 μl, was added to detect the binding of either the mono-adducts 20 and 21 or hemin to HA2. A secondary goat anti-mouse antibody conjugated with alkaline phosphatase (AP) was then added to detect the primary antibody and the binding of either the mono-adducts 20 and 21 or hemin to HA2 was recorded as optical density on a Bio-rad Microplate reader as the amount of dephosphorylation of the substrate para-nitrophenolphosphate to para-nitrophenol catalyzed by AP.

This binding curve saturated to allow the determination of the binding dissociation constant, KD₅₀, which is obtained when the rate of binding is the same as the rate of dissociation, and allows the comparison of the relative binding strengths of compounds.

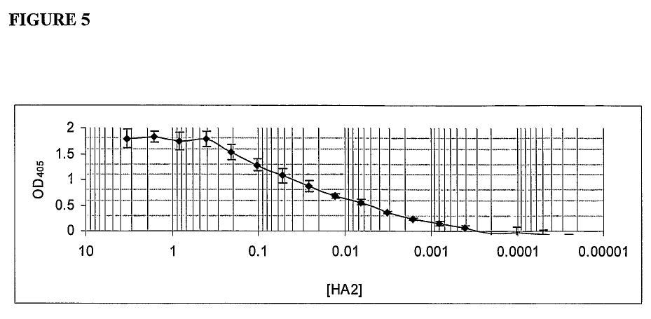

As shown in FIG. 5, it was found that the KD₅₀ of hemin to HA2 was 15+/−10 nM and the KD₅₀ of the mono-adducts 20 and 21 to HA2 was 30+/−10 nM. This shows that the mono-adducts 20 and 21 bind to HA2˜50% as strong as compared to the binding of hemin to HA2. As hemin is known to bind to HA2 very well, this shows that the mono-adducts 20 and 21 do indeed bind to HA2 and bind fairly strongly as well. This KD₅₀ was obtained from the mean of three similar KD₅₀s and may be suggesting that HA2 recognizes only one of the mono-adducts, 20 or 21, and therefore the binding strength is halved. Alternatively, it might be explained by the fact that substitution of a propionic acid moiety with metronidazole might actually result in weaker binding to HA2 which would lend weight to the hypothesis that the two propionic acid groups might be necessary for optimal binding to HA2. Separation of the mono-adducts 20 and 21 and testing them individually will resolve this point.

The microbiocidal or microbiostatic activity of the TMAs may be determined using any methods known to those of skill in the art. Exemplary methods include qualitative assays; quantitative assays; and characterization assays. Qualitative methods are used in preliminary screening of extracts or compounds for identifying the presence of constituents which inhibit bacteria and fungi, but offer little other information on these compounds. Quantitative assays (e.g. “Minimum Inhibitory Concentration Methods and the like) provides more specific information, specifically on the potency of the antimicrobial activity of compounds. Finally, techniques (such as the “Phenol Red Agar Overlay Method,” described here) which combine antimicrobial assays with techniques for analytical chemistry, allow an investigator to generate important information on the chemical nature of antimicrobial constituents which have been identified, and provide a very powerful tool for natural products chemistry.

As used herein the term “microbiostatic” refers to the ability of a compound to inhibit microbial replication, but may not involve killing of the microorganism. The term “microbiostatic” should be understood to encompass, inter alia, the meanings of the terms “bacteriostatic” and “fungistatic”. The term “microbiocidal” should be understood to refer to the ability of a compound to kill one or more microorganisms. The term “microbiocidal” should be understood to encompass, inter alia, the meanings of the terms “bactericidal” and “fungicidal”.

Exemplary antimicrobial assays, including microbiostatic and microbiocidal assays are described in detail in Murray et al. (Manual of Clinical Microbiology, American Society for Microbiology, 1999) and Reeves (Clinical Antimicrobial Assays, Oxford University Press, 1999)

In one specific embodiment of the invention a TMA comprising DPIX conjugated to metronidazole as a mono-substituted adduct, was investigated for the ability to inhibit the growth of P. gingivalis and a range of other microorganisms.

The mono-adducts 20 and 21 and metronidazole were tested on the targeted organism, P. gingivalis with the standard controls to compare the potency of the mono-adducts 20 and 21 compared to metronidazole towards the targeted organism. In a 96-well polystyrene plate divided into four sections, a series of dilutions of the mixture of the mono-adducts 20 and 21, metronidazole, DPIX 5 and DMSO were made up. These were transferred into test-tubes in quadruplicate and the P. gingivalis cells were added in triplicate leaving one test-tube as a blank control free of P. gingivalis cells. The growth of the P. gingivalis cells were monitored over 3 days (72 h) and growth was noted by the turbidity of the medium and recorded as the optical density.

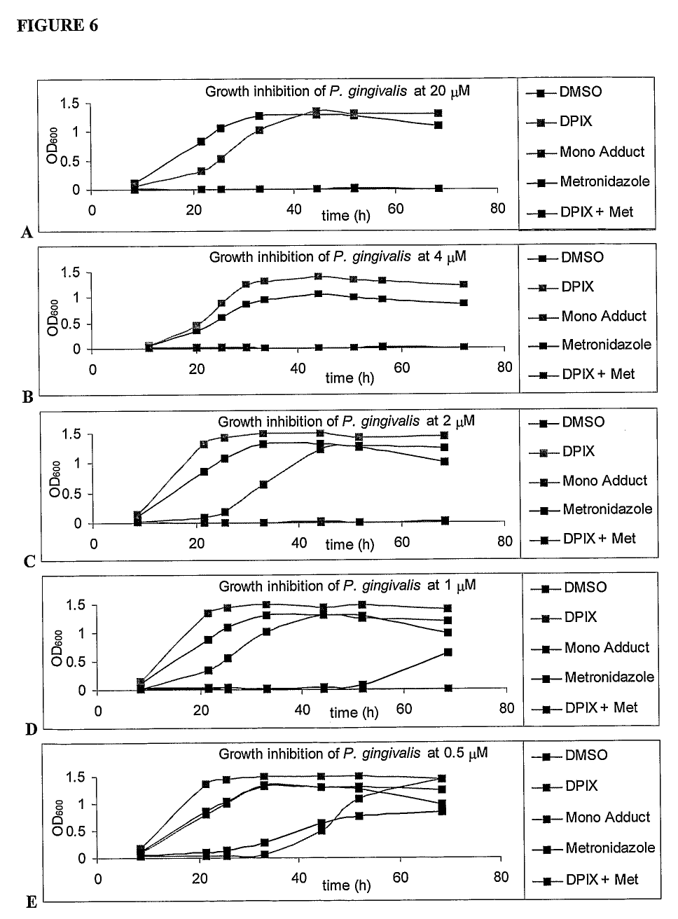

This growth inhibition assay was repeated three times and the results were shown to be consistent. The growth inhibition assays show differences in growth responses to metronidazole and the mixture of the mono-adducts 20 and 21. The results are summarized in FIG. 6.

FIG. 6A shows that there is complete inhibition of growth of P. gingivalis at 20 μM by both the mono-adducts 20 and 21 and metronidazole. This complete inhibition by both is still observed at 4 μM (FIG. 6B). However as the concentration is decreased to 2 KM (FIG. 6C), only metronidazole completely supresses growth and the mono-adducts 20 and 21 suppress growth only until about approximately 20 h, after which the mono-adducts 20 and 21 lose their activity and the P. gingivalis cells return to their original biomass and by 1 μM (FIG. 6D), metronidazole also loses its activity after approximately 50 h followed by growth recovery of the P. gingivalis cells to their original biomass. By 0.5 μM (FIG. 6E), there is no effect of growth suppression by both the mono-adducts 20 and 21 and metronidazole. From this, the Minimum Inhibitory Concentration (MIC) of the mono-adducts 20 and 21 and metronidazole can be determined to be approximately 2-4 μM and 1-2 μM respectively. This means that the mixture of the mono-adducts 20 and 21 is approximately half as potent as metronidazole.

This suggests that metronidazole is not hydrolyzing from the mixture of mono-adducts 20 and 21 in the medium and might actually be stored at the cell surface. This is because if metronidazole were hydrolyzed in the medium, there would be close to/identical effects to metronidazole as the mono-adduct comprises a 1:1 mixture of DPIX 5 and metronidazole. As the mean of the KD₅₀s from the binding assays show strong binding of the mono-adducts 20 and 21 to HA2, it might probable that at high concentrations more of the mono-adduct binds to the HA2 at the surface of the cell. This means that there is a greater likelihood that metronidazole might be hydrolyzed cell-associated esterases releasing active metronidazole for transport into the cell.

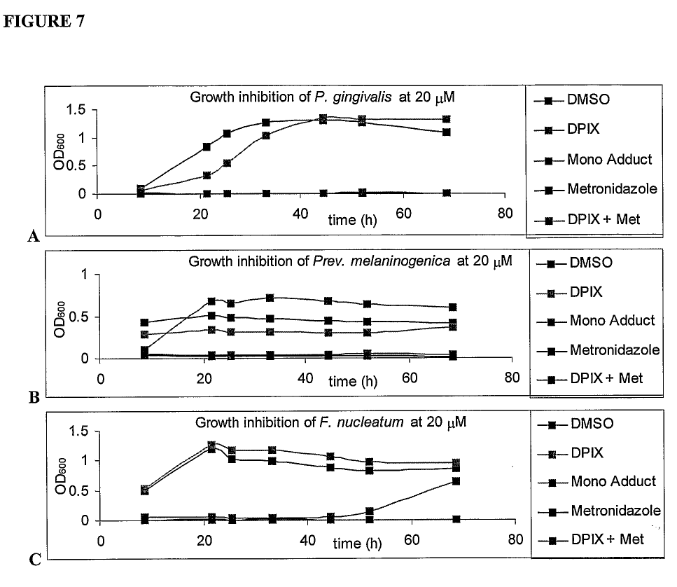

Having established that the mixture of the mono-adducts 20 and 21 bind strongly to HA2 and is approximately half as potent as metronidazole, the mono-adducts 20 and 21 and metronidazole were then tested on other strains of bacteria such as P. melaminogenica, a related organism, and F. nucleatum, an unrelated organism to assess the selectivity of the mono-adducts 20 and 21 to the targeted organism, P. gingivalis. FIG. 7 shows the growth inhibition of P. gingivalis, P. melaminogenica and F. nucleatum at 20 μM of the mono-adducts 20 and 21 with standard controls.

FIG. 7A shows complete inhibition of P. gingivalis by the mono-adducts 20 and 21 and metronidazole at 20 μM. FIG. 7B shows that there is also complete suppression of growth by metronidazole for a related organism, P. melaminogenica. However, it can be seen that the mono-adducts 20 and 21 have no effect on inhibition of P. melaminogenica at the same concentration. Instead, it seems to stimulate growth and this suggests that P. melaminogenica has other mechanisms to obtain heme.

Other targeted agents which may be conjugated to a targeting moiety would be readily ascertained by one of skill in the art and, accordingly, the present invention should not be considered in any way limited to the targeted agents recited above.

As set out supra, the targeted agent may also be a diagnostic agent or other non-cytotoxic agent. Exemplary targeted agents which may be used to generate a TMA which has diagnostic application include, but are in no way limited to optically detectable labels.

As used herein, the term “optically detectable label” refers to any molecule, atom or ion which emits fluorescence, phosphorescence and/or incandescence. The emission spectrum of the optically detectable label may be suitably chosen from the ultraviolet (wavelength range of about 350 nm to about 3 nm), visible (wavelength range of about 350 nm to about 800 nm, near infrared (NIR) (wavelength range of about 800 nm to about 1500 nm) and/or infrared (IR) (wavelength range of about 1500 nm to about 10 μm) ranges. However, due to the ease of detection, in one particularly preferred embodiment, the optically detectable label is detectable in the visible wavelength range.

In further preferred embodiments of the subject invention, the optically detectable label comprises a fluorophore.

As used herein, the term “fluorophore” refers to any molecule which exhibits the property of fluorescence. For the purposes herein, the term “fluorescence” may be defined as the property of a molecule to absorb light of a particular wavelength and re-emit light of a longer wavelength. The wavelength change relates to an energy loss that takes place in the process. The term “fluorophore” should be understood to specifically encompass, inter alia, chemical fluorophores and fluorescent dyes.

There are many fluorescent dyes that are available and which may be used as fluorophores in accordance with the present invention. An important property of a fluorescent dye or other fluorophore, which determines it's potential for use is the excitation wavelength of the fluorophore; it must match the available wavelengths of the light source. However, many different fluorescent dyes and other fluorophores will be familiar to those of skill in the art, and the choice of fluorescent marker in no way limits the subject invention.

Convenient “fluorophores” which may be used as a targeted agent in a TMA include any fluorescent marker which is excitable using a light source selected from the group below:
(i) Argon ion lasers—comprise a blue, 488 nm line, which is suitable for the excitation of many dyes and fluorochromes that fluoresce in the green to red region. Tunable argon lasers are also available that emit at a range of wavelengths (458 nm, 488 nm, 496 nm, 515 nm and others).
(ii) Diode lasers—have an emission wavelength of 635 nm. Other diode lasers which are now available operate at 532 nm. This wavelength excites propidium iodide (PI) optimally. Blue diode lasers emitting light around 476 nm are also available.
(iii) HeNe gas lasers—operate with the red 633 nm line.
(iv) HeCd lasers—operate at 325 nm.
(v) 100 W mercury arc lamp—the most efficient light source for excitation of UV dyes like Hoechst and DAPI.
(vi) Xe arc lamps and quartz halogen lamps may likewise be used as a means to excite WGMs and hence utilize the particles as sensors.

In more preferred embodiments of the present invention, the fluorescent markers are selected from: Alexa Fluor dyes; BoDipy dyes, including BoDipy 630/650 and BoDipy 650/665; Cy dyes, particularly Cy3, Cy5 and Cy 5.5; 6-FAM (Fluorescein); Fluorescein dT; Hexachlorofluorescein (HEX); 6-carboxy-4′,5′-dichloro-2′,7′-dimethoxyfluorescein (JOE); Oregon green dyes, including 488-X and 514; Rhodamine dyes, including Rhodamine Green, Rhodamine Red and ROX; Carboxytetramethylrhodamine (TAMRA); Tetrachlorofluorescein (TET); and Texas Red.

The terms “phosphorescent particles”, “phosphor particles” and “phosphors” are used interchangeably herein. What constitutes a phosphorescent optically detectable label would be readily understood by one of skill in the art. However, by way of example, which in no way limits the invention, suitable phosphors include small particles of ZnS, ZnS:Cu, Eu oxide and other phosphors used in display devices.

As used herein, the term “optically detectable label” should be understood to also encompass multiple optically detectable labels and mixtures of optically detectable labels.

As it is useful to test the mono-adducts 20 and 21 on non-related bacteria as well, as there is a large and complex community of bacteria present in the gingival crevice causing periodontal diseases, the mono-adducts 20 and 21 were also tested on the unrelated organism F. nucleatum. FIG. 7C shows complete inhibition of F. nucleatum by metronidazole at 20 μM and initial suppression of F. nucleatum by the mono-adducts 20 and 21 until approximately 45 h, after which the F. nucleatum cells return to their original biomass. This suggests, compared with the action of metronidazole, selectivity of the mono-adducts 20 and 21 for P. gingivalis compared with P. melaminogenica and F. nucleatum.

In yet another aspect, the present invention contemplates a pharmaceutical or veterinary composition comprising a TMA as described herein together with a pharmaceutically or acceptable carrier or diluent.

Composition forms suitable for injectable use include sterile aqueous solutions (where water soluble) and sterile powders for the extemporaneous preparation of sterile injectable solutions. It must be stable under the conditions of manufacture and storage and must be preserved against the contaminating action of microorganisms such as bacteria and fingi. The carrier can be a solvent or dilution medium comprising, for example, water, ethanol, polyol (for example, glycerol, propylene glycol and liquid polyethylene glycol, and the like), suitable mixtures thereof and vegetable oils. The proper fluidity can be maintained, for example, by the use of superfactants. The preventions of the action of microorganisms can be brought about by various anti-bacterial and anti-fungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with the active ingredient and optionally other active ingredients as required, followed by filtered sterilization or other appropriate means of sterilization. In the case of sterile powders for the preparation of sterile injectable solutions, suitable methods of preparation include vacuum drying and the freeze-drying technique which yield a powder of active ingredient plus any additionally desired ingredient.

When the targeted agent is suitably protected, it may be orally administered, for example, with an inert diluent or with an assimilable edible carrier, or it may be enclosed in hard or soft shell gelatin capsule, or it may be compressed into tablets, or it may be incorporated directly with the food of the diet or administered via breast milk. For oral therapeutic administration, the active ingredient may be incorporated with excipients and used in the form of ingestible tablets, buccal tablets, troches, capsules, elixirs, suspensions, syrups, wafers and the like. Such compositions and preparations should contain at least 1% by weight of targeted agent. The percentage of the compositions and preparations may, of course, be varied and may conveniently be between about 5 to about 80% of the weight of the unit. The amount of targeted agent in such therapeutically useful compositions is such that a suitable dosage will be obtained. Preferred compositions or preparations according to the present invention are prepared so that an oral dosage unit form contains between about 0.1 μg and 200 mg of modulator. Alternative dosage amounts include from about 1 μg to about 1000 mg and from about 10 μg to about 500 mg. These dosages may be per individual or per kg body weight. Administration may be per hour, day, week, month or year.

The tablets, troches, pills, capsules and the like may also contain the components as listed hereafter. A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the modulator, their use in the therapeutic compositions is contemplated. Supplementary active compounds can also be incorporated into the compositions.

Another aspect of the present invention contemplates a method for the prophylaxis or treatment of infection by a microorganism with one or more auxotrophic requirements in a biological environment from where the microorganism acquires said auxotrophic requirement, said method comprising administering to said environment an effective amount of a TMA as described herein for a time and under conditions sufficient to have a microbiocidal or microbiostatic effect on said microorganism.

The term “biological environment” is used in its broadest context to include any environment from which a target microorganism acquires one or more auxotrophic requirements. Preferably, the biological environment is on or within a cell, tissue or organ of an animal subject such as a mammal, reptile, amphibian, fish or bird or is a hoof of a livestock animal. More preferably, the animal is a mammal such as a human or livestock animal.

In one preferred embodiment of the invention, porphyrin, a porphyrin analog or a porphyrin-like molecule is an auxotrophic requirement of the target microorganism. Even more preferably, heme is an auxotrophic requirement of said microorganism.

Accordingly, another aspect of the present invention contemplates a method for the prophylaxis or treatment of infection by a microorganism, for which heme is an auxotrophic requirement, in a biological environment from where the microorganism acquires said heme, said method comprising administering to said environment an effective amount of a TMA comprising porphyrin, a porphyrin analog or a porphyrin-like molecule as a targeting moiety for a time and under conditions sufficient to have a microbiocidal or microbiostatic effect on said microorganism.

Although not intending to limit the present invention to any one theory or mode of action, it is proposed that P. gingivalis and its relatives do not have a complete functional porphyrin-synthesizing pathway and hence are porphyrin auxotrophs. In particular, it is proposed that P. gingivalis lacks one or more of a glutamyl-t RNA reductase, porphobilinogen synthase, porphobilinogen deaminase, uroporphyrinogen III cosynthase, uroporphyrinogen decarboxylase, coproporphyrinogen III oxidase, HemM or uroporphyrinogen III methylase. As a result, P. gingivalis needs to acquire porphyrin for growth and/or maintenance or at least to facilitate growth and/or maintenance.

Accordingly, in a particularly preferred embodiment, the present methods are adapted to the treatment of P. gingivalis infections in the oral cavity of an animal subject.

Although the method of the present invention is particularly directed to P. gingivalis infection in the oral cavity such as during periodontal disease, it extends to any disease condition resulting from microbial infection and in particular infection by P. gingivalis or any other microorganism which comprises an auxotrophic requirement for heme, porphyrin, a porphyrin analog, a porphyrin-like molecule. Examples of microorganisms contemplated herein include but are not limited to Salmonella spp., Serratia spp., Yersinia spp., Klebsiella spp., Vibrio spp., Pseudomas spp., E. coli, Haemophilus spp. Examples of P. gingivalis or related microorganism infection contemplated by the present invention include infection of the oral cavity, nasopharynx, oropharynx, vagina and urethra as well as infection of mucous membranes and infection of hooves of livestock animals such as sheep, cattle and goats.

An “effective” amount means a sufficient amount to have a microbiocidal or microbiostatic effect on the target organism.

A related aspect of the present invention contemplates a method for prophylaxis or treatment of periodontal, pulmonary, vaginal, urethral or hoof disease resulting from infection by P. gingivalis or related microorganism in a mammal, said method comprising administering to said mammal an effective amount of a TMA as described herein for a time and under conditions sufficient to have a microbiocidal or microbiostatic effect on the microorganism.

The term “infection” is used in its most general sense and includes the presence, reproduction or growth of a microorganism resulting in a disease condition or having the capacity to result in a disease condition. The term “infection” further encompasses P. gingivalis or other microorganisms when present as part of the normal flora. Such bacteria may, under certain circumstances, be responsible for disease development. Prophylaxis is contemplated in accordance with the present invention to reduce the levels of P. gingivalis or related microorganism or to reduce the likelihood of a disease condition developing resulting from infection by P. gingivalis or a relative thereof.

The present invention is particularly directed to the treatment of P. gingivalis or a related microorganism in humans. The present invention extends, however, to the prophylaxis or treatment of P. gingivalis or related microorganisms in other mammals such as primates, livestock animals (e.g. sheep, cows, goats, pigs, horses, donkeys), companion animals (e.g. dogs, cats), laboratory test animals (e.g. mice, rats, guinea pigs, rabbits, hamsters) and captured wild animals.

In a related aspect of the present invention, there is provided a use of a TMA in the manufacture of a medicament for the prophylaxis or treatment of infection by a target microorganism comprising one or more auxotrophic requirements.

Preferably, the target microorganism requires heme, porphyrin, a porphyrin analog or a porphyrin-like molecule as an auxotrophic requirement. More preferably, the target microorganism is P. gingivalis.

In yet another aspect, the present invention provides a method of enumerating, visualising or localising a subject organism comprising one or more auxotrophic requirements, said method comprising administering to said organism or the environment of said organism a TMA of any one of general Formulae (I), (II) or (III), wherein the targeted agent A, is an optically detectable label, and detecting said optically detectable label, wherein detection of the optically detectable label is indicative of the presence, location and/or amount of the organism.

In one preferred embodiment, the auxotrophic requirement of the organism comprises porphyrin, a porphyrin analog or a porphyrin-like molecule.

In another preferred embodiment, the method is used to enumerate, visualize or localise P. gingivalis or a related organism.

In yet another aspect, the method of the present invention may be used to diagnose infection by a subject microorganism with one or more auxotrophic requirements in a biological environment from where the microorganism acquires said auxotrophic requirement, said method comprising administering to said biological environment a TMA of any one of general Formulae (I), (II) or (III), wherein the active agent, A is an optical detectable label, wherein the presence of the subject microorganism is indicated by the TMA.

In one preferred embodiment, the method is used to diagnose infections caused by a microorganism with an auxotrophic requirement for porphyrin, a porphyrin analog or a porphyrin-like molecule. In a further preferred embodiment, the method is used to diagnose infection by P. gingivalis or a related organism.

In another preferred embodiment, the method is used to diagnose infections in the oral cavity of a human or other animal subject.

The present invention is further described by the following non-limiting Examples.

### EXAMPLE 1

### General Procedures

Melting points were recorded on a Reichert melting point stage microscope or a Gallenkamp melting point apparatus and are uncorrected.

¹H Nuclear Magnetic Resonance (¹H NMR) spectra were recorded on a Bruker Avance DPX 200 spectrometer at a frequency of 200.13 MHz or a Bruker Avance DPX 300 spectrometer at a frequency of 300.13 MHz at 300 K. Samples were dissolved in deuterated chloroform (CDCl₃) containing tetramethylsilane (TMS) as an internal reference, unless otherwise stated. The signals are recorded in terms of chemical shift (δ in ppm) relative to TMS (SiMe₄, ¹H=0 ppm) or the solvent residue peak (CDCl₃, ¹H=7.26 ppm), unless otherwise stated, relative integral, multiplicity (s, singlet; br, broad singlet; d, doublet; t, triplet; m, multiplet; dd, doublet of doublets; dt, doublet of triplets), coupling constants (J in Hz) and assignments, in that order. The numbering system employed in the presentation of ¹H NMR data for the porphyrins depicted in this thesis is described in Appendix A1. NMR data were processed on Silicon Graphics Industries (SGI) and PC workstations using standard Bruker software (xwinNMR).

Matrix Assisted Laser Desorption Ionisation-Time of Flight (MALDI-TOF) mass spectra were recorded on a Micromass T of Spec 2E spectrometer. Mass spectra were recorded without matrix. Mass spectra were obtained as an envelope of the isotope peaks of the molecular ion. The mass corresponding to the maxima of the envelope was reported and compared with the maxima of a stimulated spectrum.

Low-resolution ElectroSpray Ionization (ESI) mass spectra were recorded on a ThermoQuest Finnigan LCQ Deca ion trap mass spectrometer. Instrument was controlled and data collected using Xcalibur software. High-resolution mass spectroscopy was performed on a Bruker Fourier Transform Ion Cyclotron Resonance (FTICR) mass spectrometer in electrospray mode with a 4.7 T superconducting magnet by Dr. Keith Fisher at the Australian National University, Canberra, Australia.

Infrared absorption spectra were recorded on a Perkin-Elmer Model 1600 FTIR spectrophotometer or a Shimadzu Model 8400 FTIR spectrophotometer as solutions in the stated solvents. Intensity abbreviations are used: w, weak; m, medium; s, strong.

Electronic absorption spectra were carried out on a Cary 1E UV-Vis spectrophotometer or a Cary 5E UV-Vis spectrophotometer using the solvent stated at 298 K and approximately 10⁻⁵ M solutions.

Preparative column chromatography was carried out routinely on Merck Kieselgel 60 silica gel (SiO₂, 0.040-0.063 mm). All columns were run in dim light (aluminum foil) using redistilled solvents. Analytical thin layer chromatography (TLC) was performed using Merck Kieselgel silica gel 60 F-254 precoated sheets (0.2 mm). Ratios of solvents system for column chromatography and TLC were expressed as v/v as specified.

Analytical and preparative reverse phase HPLC was carried out on a Waters 600E solvent delivery system with a Rheodyne 7125 injector and a Waters 486 UV detector using the solvent system CH₃OH:CH₃CN:CF₃COOH (50:50:0.05). Instruments were controlled and data collected using Millennium software.

Reagents were commercially available reagent grade chemicals and most reagents were used without further purification. Resorcinol was recrystallized from dichloromethane. Pyridine was dried by distilling over calcium hydride. All commercial solvents were purified according to literature methods and routinely distilled before use. Ether refers to diethyl ether and light petroleum refers to the fraction with a boiling point of 60-80° C. Ethanol-free chloroform was obtained by drying distilled chloroform over calcium chloride and passing it through a column of neutral alumina immediately prior to use. Deuterated chloroform was deacidified before use in NMR spectroscopy by passing through a column of neutral alumina. Deuterated solvents were purchased from Aldrich, Merck and Cambridge Isotope Laboratories.

Bovine hemoglobin (Hb), Hb-agarose beads, hemin (Hm) and Hm-agarose beads were supplied by Sigma-Aldrich Company. The translated product of a synthetic HA2 gene, derived from the sequence of RgpA, was expressed in E. coli with a C-terminal polyhistidine tag (International Patent Application No. WO 2002061091) and is referred to as rHA2. rHA2 was functionally purified by Hb-agarose affinity chromatography. Anti-gingipain monoclonal antibody (mAb) 5A1 was prepared in mice. Secondary goat anti-mouse antibody conjugated with alkaline phosphatase (AP) was supplied by Dako Corporation. 4-nitrophenylphosphate (p-NPP) was supplied by Boehringer. P. gingivalis ATCC 33277 cells was used in all growth assays and cultures were anaerobically grown at 37° C. in an atmosphere containing CO₂ (5%), H₂ (10%) and N₂ (85%). P. gingivalis and P. melaminogenica cells were grown in Center for Disease Control (CDC) medium and F. nucleatum cells were grown in Brain Heart Infusion (BHI) medium.

### EXAMPLE 2

### Synthesis of Deuterohemin

### Method 1:

Following the method of Smith, 1999 supra; 1976 supra, protohemin (10.1 g, 15.5 mmol) was ground with resorcinol (30.2 g, 274 mmol) and heated at reflux at 160° C. under an air condenser for 1 h to yield crude deuterohemin. The reaction mixture was filtered and washed with ether until the washings were colourless to yield deuterohemin 23 (9.12 g, 98.1%) as a black solid, m.p.>300° C.; m/z (MALDI-TOF) 564.2 [(M)⁺ requires 564.4].

### Method 2:

Following the method of Adler et al. (supra 1977) protohemin (5.00 g, 7.67 mmol) was ground with resorcinol (25.0 g, 227 mmol) and heated at reflux at 190° C. under an air condenser for 25 min. Care was taken to prevent the temperature from rising above 200° C. After the melt had cooled to 140° C., hot propionic acid (50 ml) was added with stirring and the resulting solution was poured into water (500 ml). The solution was neutralized with sodium hydroxide (10 M, 950 ml) and heated for 1 h. The solution was allowed to cool and left for 12 h to allow the fine precipitate to aggregate. The solution was vacuum filtered with a large sintered funnel as the filtration is slow and the filtrate was filtered until clear. The precipitate was air-dried and dissolved in methanol (100 ml). The solution was filtered and the solvent removed. The residue was transferred to a vacuum desiccator and left for 12 h in vacuo to afford crude deuterohemin 23. Water was added with heating and the solution was filtered. The solvent was removed to yield deuterohemin 23 (3.51 g, 76.3%) as a black solid with an identical mass spectrum to the product obtained by Method 1.

### EXAMPLE 3

### Synthesis of 1,3,5,8-tetramethylporphyrin-6,7-dipropanoic acid dimethyl ester-Deuteroporphyrin IX dimethyl ester (DPIX DME) 24

Following the method of Smith, 1999 supra; 1976 supra, deuterohemin 23 (200 mg, 0.333 mmol) was dissolved in glacial acetic acid (50 ml) and hydrochloric acid (10 M, 2 ml), iron powder (50.2 mg, 0.899 mmol) was added and the reaction mixture was heated at reflux at 160° C. for 30 min. The cooled solution was diluted with water (52 ml), treated with a saturated sodium acetate solution (80 ml) and extracted with ethyl acetate (2×100 ml). The combined ethyl acetate layers were extracted with hydrochloric acid (1.5 M, 2×100 ml). The aqueous layers were combined, adjusted to pH 4 with sodium hydroxide (3 M) and extracted into ethyl acetate (3×100 ml). The solvent was removed, and a mixture of methanol (200 ml) and sulfuric acid (10 ml) was added and the solution was left overnight. The solution was adjusted to pH 4 with sodium hydroxide (3 M) and extracted with ethyl acetate. The porphyrin in the ethyl acetate layer was acidified with hydrochloric acid (1.5 M) and the layers were separated. The porphyrin was extracted from the aqueous phase with chloroform (3×50 ml) after adjusting the pH of the acid extracts to pH 4 with sodium hydroxide. The chloroform extract was washed with water, dried over sodium sulfate and the solvent was removed. The crude product was chromatographed on a neutral alumina column with an eluent of 2% v/v methanol/chloroform. Evaporation of the purple-red elutes yielded deuteroporphyrin IX dimethyl ester 24 (110 mg, 61.3%) as a purple-red solid, m.p. 219-220° C. (lit. m.p. 217-220° C.) with an identical ¹H NMR spectrum to that quoted in the literature.

λₘₐₓ (CHCl₃)/nm 399 (log ε 5.24), 497 (4.13), 529 (4.00), 565 (3.91), 620 (3.69); ¹H NMR (200 MHz; CDCl₃; SiMe₄) δ_{H} −4.04 (2H, s, inner NH), 3.21-3.28 (4H, t, 7.3 Hz, —CH₂CH₂CO₂CH₃), 3.57 (3H, s, —CH₃), 3.63 (3H, s, —CH₃), 3.64 (6H, s, —CH₂CH₂CO₂CH₃), 3.67 (3H, s, —CH₃), 3.68 (3H, s, —CH₃), 4.32-4.39 (4H, t, 7.7 Hz, —CH₂CH₂CO₂CH₃), 9.02 (2H, s, β-pyrrolic H₂H₄), 9.91 (1H, s, γ-H), 9.94 (1H, s, δ-H), 9.98 (1H, s, α-H), 10.01 (1H, s, β-H); m/z (MALDI-TOF) 539.3 [(M+H)⁺ requires 539.7].

### EXAMPLE 4

### Synthesis of 1,3,5,8-tetramethyl-porphine-6,7-dipropanoic acid-Deuteroporphyrin IX (DPIX) 5

### Method 1: The Formic Acid Method

Deuterohemin 23 (2.01 g, 3.35 mmol) and formic acid (80 ml) were heated at reflux under nitrogen and stirred vigorously in a 100 ml 3-neck round bottom flask, fitted with a mechanical stirrer. Iron powder (2.00 g, 35.8 mmol) was added in 500 mg portions at intervals of 5 min and the reaction mixture was followed by TLC at 5 min intervals to check the progress of the reaction which was completed after 20 min. The reaction mixture was poured into water (100 ml) and the resulting mixture was treated with a saturated sodium acetate solution (80 ml) and extracted with ethyl acetate (4×100 ml). The combined ethyl acetate layers were extracted with hydrochloric acid (1.5 M, 3×100 ml) and the aqueous layers were combined and adjusted to pH 4 with sodium hydrogen carbonate. The porphyrin was extracted from the aqueous phase with ethyl acetate (4×200 ml) and the solvent was removed to yield deuteroporphyrin IX 5 (1.30 g, 76.0%) as purple-black crystals, m.p.>300° C. with an identical ¹H NMR spectrum to that quoted in the literature.

λₘₐₓ (Acetone)/nm 394 (log ε 5.18), 495 (4.23), 524 (4.09), 565 (4.05), 619 (3.99); ¹H NMR (200 MHz; Acetone-D₆; SiMe₄) δ_{H} −4.13 (2H, s, inner NH), 3.41-3.48 (4H, t, 7.6 Hz, —CH₂CH₂CO₂H), 3.65 (3H, s, —CH₃), 3.68 (3H, s, —CH₃), 3.74 (3H, s, —CH₃), 3.77 (3H, s, —CH₃), 4.43-4.50 (4H, t, 7.8 Hz, —CH₂CH₂CO₂H), 9.11 (2H, s, β-pyrrolic H₂H₄), 10.05-10.17 (4H, m, meso H, γ-, δ-, α-, β-H); m/z (MALDI-TOF) 511.3 [(M+H)⁺ requires 511.6]; m/z (FTICR-MS) 511.2320; [(M+H)⁺, calcd for [C₃₀H₃₀N₄O₄+H⁺]: 511.2339].

### Method 2: The Iron Powder Method

### Work-Up 1 Using Ethyl Acetate as Solvent for Extraction

Following the method of Smith, 1999 supra; 1976 supra, deuterohemin 23 (400 mg, 0.667 mmol) was dissolved in glacial acetic acid (100 ml) and hydrochloric acid (10 M, 2 ml), iron powder (100 mg, 1.79 mmol) was added and the reaction mixture was heated at reflux at 150° C. for a further 30 min. The cooled solution was diluted with water (102 ml), mixed with saturated sodium acetate (160 ml) and extracted with ethyl acetate (4×60 ml). The combined ethyl acetate layers were extracted with hydrochloric acid (1.5 M, 2×100 ml). The aqueous layers were combined, adjusted to pH 4 with sodium hydroxide (3 M) and extracted into ethyl acetate (3×100 ml). The solvent was removed to yield deuteroporphyrin IX 5 (208 mg, 61.1%) as a purple-red solid, with an identical ¹H NMR and mass spectrum to the product obtained by Method 1.

### Work-Up 2 Using Dichloromethane as Solvent for Extraction

Following the method of Smith, 1999 supra; 1976 supra, deuterohemin 23 (1.02 g, 1.70 mmol) was dissolved in glacial acetic acid (400 ml) and hydrochloric acid (10 M, 5 ml), iron powder (1.01 g, 18.1 mmol) was added and the reaction mixture was heated at reflux at 160° C. for a further 30 min. The cooled solution was diluted with water (405 ml), mixed with saturated sodium acetate (640 ml) and extracted with dichloromethane (3×450 ml). The combined dichloromethane layers were extracted with hydrochloric acid (1.5 M, 3×300 ml). The aqueous layers were combined, adjusted to pH 4 with a sodium hydrogen carbonate solution and extracted into dichloromethane (5×300 ml). The solvent was removed to yield deuteroporphyrin IX 5 (106 mg, 12.2%) as a purple-red solid, with an identical ¹H NMR and mass spectrum to the product obtained by Method 1.

### Work-Up 3 Using N-Butanol as Solvent for Extraction

Following the method of Smith, 1999 supra; 1976 supra, deuterohemin 23 (100 mg, 0.167 mmol) was dissolved in glacial acetic acid (50 ml) and hydrochloric acid (10 M, 2 ml), iron powder (140 mg, 2.51 mmol) was added and the reaction mixture was heated at reflux at 170° C. under nitrogen for 40 min. The reaction mixture was followed by TLC. The cooled solution was diluted with water (52 ml), mixed with saturated sodium acetate (80 ml) and extracted with dichloromethane (4×100 ml). The combined dichloromethane layers were extracted with hydrochloric acid (1.5 M, 2×300 ml). The aqueous layers were combined, adjusted to pH 4 with a sodium hydrogen carbonate solution and extracted into dichloromethane (5×300 ml). The aqueous layer was extracted into n-butanol (1×200 ml). The solvent was removed to yield deuteroporphyrin IX 5 (10.5 mg, 12.3%) as a purple-red solid, with an identical ¹H NMR and mass spectrum to the product obtained by Method 1.

### Method 3: The Ferrous Sulfate Method

Following the method of Smith, 1999 supra; 1976 supra, deuterohemin 23 (500 mg, 0.834 mmol) was dissolved in pyridine (5 ml) and methanol (45 ml). Ferrous sulfate (500 mg) was added and dry hydrogen chloride gas was passed rapidly through the solution. The solution was diluted with water (100 ml), mixed with saturated sodium acetate (160 ml) and extracted with ethyl acetate (4×60 ml). The combined ethyl acetate layers were extracted with hydrochloric acid (1.5 M, 2×100 ml). The aqueous layers were combined, adjusted to pH 4 with sodium hydroxide (3 M) and extracted into ethyl acetate (3×100 ml). The solvent was removed to yield deuteroporphyrin IX 5 (70.8 mg, 16.6%) as a purple-red solid, with an identical ¹H NMR and mass spectrum to the product obtained by Method 1.

Method 4: De-esterification of 1,3,5,8-tetramethylporphyrin-6,7-dipropanoic acid dimethyl ester-Deuteroporphyrin IX dimethyl ester (DPIX DME) 24

Following the method of Smith, 1999 supra; 1976 supra, deuteroporphyrin IX dimethyl ester 24 (50.2 mg, 0.0931 mmol) was dissolved in a solution (25 ml) of potassium hydroxide (1.01 g, 18.0 mmol), water (95 ml) and methanol (5 ml). The mixture was extracted with ethyl acetate. The aqueous layer was extracted with ethyl acetate-water (1:1, 150 ml) and acidified to pH 4. The ethyl acetate extract was dried with anhydrous sodium sulphate, filtered and the solvent removed to yield deuteroporphyrin IX 5 (18.4 mg, 38.7%) as a purple-red solid, with an identical ¹H NMR and mass spectrum to the product obtained by Method 1.

### EXAMPLE 6

### Attempted Synthesis of Deuteroporphyrin IX-TMS Adducts

Following the method of Royappa et al. Langmuir 14:6207-6214, 1998, deuteroporphyrin IX 5 (30.0 mg, 0.0588 mmol) was dissolved in anhydrous ether. Dry pyridine (9.3 mg, 0.12 mmol) was added, and the reaction flask was purged with nitrogen. Trimethylsilyl chloride (13.0 mg, 0.119 mmol) was added over 30 s while the reaction mixture was vigorously stirred. The reaction was stirred over nitrogen for 3 h. The solvent was then removed under vacuum to yield a purple-red solid. Both mass spectra (MALDI-TOF and ESI) and ¹H NMR (200 MHz; CDCl₃; SiMe₄) spectroscopy of the product suggested that the deuteroporphyrin IX-TMS adducts were not obtained and showed that no reaction had occurred.

### EXAMPLE 7

### Synthesis of 2,4-diethenyl-1,3,5,8-tetramethyl-porphine-6,7-dipropanoic acid dimethyl ester-Protoporphyrin IX dimethyl ester 26 (PPIX DME)

Following the method of Smith, 1999 supra; 1976 supra, hematoporphyrin IX 25 (500 mg, 0.744 mmol) and p-toluenesulfonic acid (1.25 g, 6.47 mmol) were heated at reflux at 140° C. under N₂ gas in chlorobenzene (250 ml) for 2 h. The cooled solution was shaken with ammonia (128 ml), glacial acetic acid (50 ml) was added and the organic layer was separated. The aqueous layer was extracted with ethyl acetate (2×50 ml) and the organic extracts were combined, dried over anhydrous sodium sulfate, filtered and the solvent was removed. Methanol (200 ml) and sulfuric acid (18 M, 10 ml) were added to the residue and the reaction mixture was left in the dark overnight at room temperature. The solution was diluted with water (500 ml) and extracted with dichloromethane (2×80 ml). The combined organic extracts were washed with sodium hydrogen carbonate, dried over anhydrous sodium sulfate, filtered and the solvent was removed. The crude product was chromatographed on a silica column with an eluent of 2% v/v methanol/chloroform. Evaporation of the solution yielded protoporphyrin IX dimethyl ester 26 (365 mg, 83.1%) as a dark red solid, m.p. 198-202° C. with an identical ¹H NMR spectrum to that quoted in the literature.

λₘₐₓ(CHCl₃)/nm 408 (log ε 5.15), 506 (3.89), 541 (3.76), 576 (3.38) and 630 (3.05); ¹H NMR (200 MHz; CDCl₃; SiMe₄) δ_{H} −3.62 (2H, s, inner NH), 3.25-3.33 (4H, t, 7.7 Hz, —CH₂CH₂CO₂CH₃), 3.63 (3H, s, —CH₃), 3.65 (3H, s, —CH₃), 3.66 (6H, s, —CH₃), 3.72 (3H, s, —CH₃), 3.74 (3H, s, —CH₃), 4.38-4.45 (4H, t, 7.6 Hz, —CH₂CH₂CO₂CH₃), 6.20 (2H, dt, 11.5 Hz and 1.6 Hz, —CH═CH_{A}H_{B}), 6.40 (2H, dt, 17.9 Hz and 1.7 Hz, —CH═CH_{A}H_{B}), 8.26 (1H, dd, 16.9 Hz and 11.7 Hz, —CH═CH_{A}H_{B}), 8.34 (1H, dd, 16.1 Hz and 11.6 Hz, —CH═CH_{A}H_{B}), 10.06 (1H, s, γ-H), 10.12 (1H, s, δ-H), 10.20 (1H, s, α-H), 10.26 (1H, s, β-H); m/z (MALDI-TOF) 591.5 [(M+H)⁺ requires 591.7].

### EXAMPLE 8

### Synthesis of 2,4-diethenyl-1,3,5,8-tetramethyl-porphine-6,7-dipropanoic acid-Protoporphyrin IX (PPIX) 3

Following the method of Smith, 1999 supra; 1976 supra, protoporphyrin IX dimethyl ester 26 (100 mg, 0.169 mmol) was dissolved in a solution of potassium hydroxide (3.02 g, 53.8 mmol) and methanol (35 ml) and heated at reflux under N₂ gas in the dark overnight. Upon cooling, the solution was extracted with ethyl acetate. The combined ethyl acetate layers were extracted with hydrochloric acid (3 M, 2×50 ml). The aqueous layers were combined, adjusted to pH 4 with sodium hydroxide (3 M) and extracted into ethyl acetate (3×100 ml). The ethyl acetate layer was dried over anhydrous sodium sulfate, filtered and the solvent removed to yield pure protoporphyrin IX 3 (72.8 mg, 76.6%) as a purple-red solid, m.p.>300° C. with an identical ¹H NMR spectrum to that quoted in the literature.

λₘₐₓ(CHCl₃/TFA)/nm 413 (log ε 5.24), 525 (3.25), 556 (3.99), 578 (3.57) and 600 (3.63); ¹H NMR (200 MHz; CDCl₃; SiMe₄) δ_{H} −3.33 (2H, s, inner NH), 3.19-3.26 (4H, t, 6.6 Hz, —CH₂CH₂CO₂H), 3.63 (3H, s, —CH₃), 3.67 (3H, s, —CH₃), 3.69 (3H, s, —CH₃), 3.72 (3H, s, —CH₃), 4.48 (4H, t, 5.1 Hz, —CH₂CH₂CO₂H), 6.30 (2H, dd, 17.8 Hz and 10.0 Hz, —CH═CH_{A}H_{B}), 6.47 (2H, dd, 11.4 Hz and 3.1 Hz, —CH═CH_{A}H_{B}), 8.09-8.27 (2H, m, —CH═CH_{A}H_{B}), 10.63 (1H, s, γ-H), 10.65 (1H, s, δ-H), 10.90 (1H, s, α-H), 10.92 (1H, s, β-H); m/z (MALDI-TOF) 563.4 [(M+H)⁺ requires 563.7].

### EXAMPLE 9

### Attempted Synthesis of Copper(II) Hematoporphyrin IX 27

Following the method of Adler, 1970 supra hematoporphyrin IX 25 (200 mg, 0.298 mmol) and copper(II) acetate (100 mg, 0.669 mmol) were dissolved in dimethylformamide (80 ml) and the solution was heated at reflux for 3 h. Upon cooling, the solvent was removed to yield black crystals. Both MALDI-TOF and ESI mass spectra suggested that copper(II) hematoporphyrin IX 27 was not obtained and showed that no reaction had occurred.

### EXAMPLE 10

### Synthesis of Deuteroporphyrin IX di- & Mono-Substituted Adducts

### Method 1 Via Deuteroporphyrin IX Di [Acid Chloride] 31

Deuteroporphyrin IX 5 (200 mg, 0.392 mmol) was refluxed in a mixture of thionyl chloride (10 ml) and dichloromethane (40 ml) under nitrogen for 30 min. Upon cooling, the solvent was removed. The residue was dissolved in dichloromethane (3×20 ml) and the solvent removed after each addition of dichloromethane to remove any trace of thionyl chloride. Dichloromethane (50 ml) was added to the resulting residue and metronidazole (26.8 mg, 0.157 mmol) was added to the solution along with triethylamine (5 drops). The solution was refluxed under nitrogen for 1 h and upon cooling the solvent was removed. Toluene (20 ml) was added and evaporated. A mixture of toluene (20 ml) and water (0.5 ml) was added and the two-phase mixture was stirred for 30 min. The solvent was removed to yield a mixture of products as a reddish-black solid.

The mixture was passed through a silica column with an eluting solvent of a 30:1:1 mixture of CH₂Cl₂:CH₃OH:CH₃NO₂. The eluting solvent was changed to 20:1:1 upon which the first band eluted. The polarity of the eluting solvent was then increased to 10:1:1 upon which the second band eluted.

The fractions which contained the first band were evaporated to dryness to yield the di-metronidazole-substituted adduct 19 (11.8 mg, 3.7%) as a dark red solid, m.p. 90° C.

νₘₐₓ (CHCl₃)/cm⁻¹ 3316w (inner NH), 2992m, 2983m, 2917m, 2900m, 2147m, 2064w, 1737s (C═O), 1661s (NO₂C═CH), 1653s (NO₂C═CH), 1528w, 1465w, 1364w; λₘₐₓ (CHCl₃)/nm 400 (log ε 5.10), 497 (3.99), 530 (3.72), 566 (3.63) and 620 (3.40); ¹H NMR (300 MHz; CDCl₃; SiMe₄) δ_{H} −4.04 (2H, s, inner NH), 1.54 (3H, s, N═C—CH₃), 1.56 (3H, s, N═C—CH₃), 3.13-3.21 (4H, t, 7.4 Hz, —CH₂), 3.56 (3H, s, —CH₃), 3.57 (3H, s, —CH₃), 3.70 (3H, s, —CH₃), 3.72 (3H, s, —CH₃), 3.72-3.86 (4H, m, —CH₂), 4.07-4.14 (4H, m, —CH₂), 4.28-4.35 (4H, t, 6.0 Hz, —CH₂), 7.42 (1H, s, NO₂C═CH), 7.47 (1H, s, NO₂C═CH), 9.05 (2H, s, β-pyrrolic H₂H₄), 9.91 (1H, s, γ-H), 9.96 (1H, s, δ-H), 10.03 (1H, s, α-H), 10.05 (1H, s, β-H); m/z (MALDI-TOF) 817.7 [(M+H)⁺ requires 817.9]; m/z (FTICR-MS) 817.3349; [(M+H)⁺, calcd for [C₄₂H₄₄N₁₀O₈+H⁺]: 817.3416].

The fractions which contained the second band were evaporated to dryness to yield the mixture of mono-metronidazole-substituted adducts 20 and 21 (26.2 mg, 8.2%) as a dark red solid, m.p. 70° C.

νₘₐₓ (CHCl₃)/cm⁻¹ 3317w (inner NH), 3136w (O═C—OH), 3005w, 2980w, 2922w, 2471w, 1736s (C═O), 1562s (NO₂C═CH), 1531m, 1468s, 1427s, 1381m, 1366s; λₘₐₓ (CHCl₃)/nm 400 (log ε 5.11), 497 (3.99), 531 (3.70), 566 (3.59) and 620 (3.36); ¹H NMR (300 MHz; CDCl₃; SiMe₄) δ_{H} −4.32 (2H, s, inner NH), 0.77-0.79 (3H, s, N═C—CH₃), 3.12 (2H, broad t, —CH₂), 3.21-3.27 (2H, t, 6.4 Hz, —CH₂), 3.43-3.65 (12H, m, —CH₃), 3.90-3.97 (4H, m, —CH₂), 4.04-4.28 (4H, m, —CH₂), 7.12 (1H, s, NO₂C═CH), 8.92-8.96 (2H, d, β-pyrrolic H₂H₄), 9.81-9.91 (4H, m, meso H); m/z (MALDI-TOF) 664.5 [(M+H)⁺ requires 664.7]; m/z (FTICR-MS) 664.2869; [(M+H)⁺, calcd for [C₃₆H₃₇N₇O₆+H⁺]: 664.2879].

### Method 2 Via Deuteroporphyrin IX Di-Anhydride 30

Deuteroporphyrin IX 5 (56.0 mg, 0.110 mmol) was refluxed in acetic anhydride (28.0 μl, 0.297 mmol) and toluene (40 ml) for 4 h. The mixture was filtered while hot, then allowed to cool. The acetic anhydride was removed under high vacuum at room temperature to give crude deuteroporphyrin IX di-anhydride 30. The crude product 30 and metronidazole (14.5 mg, 0.0847 mmol) were heated at reflux in toluene (12 ml) under nitrogen for 11 h 30 min. The solvent was removed under high vacuum to yield a mixture of products 5, 19, 20 and 21 (28.6 mg) as a reddish-black solid, with an identical ¹H NMR and mass spectrum to the products obtained by Method 1.

### EXAMPLE 11

### Synthesis of N-pyridinium sulfonic acid

Following the method of Smith, 1999 supra; 1976 supra, pyridine (8 ml) was dissolved in chloroform (24 ml) and the solution was stirred mechanically in an ice-salt bath. Chlorosulfonic acid (3 ml) was added dropwise in a fume cupboard and the thick white precipitate is filtered, washed with water at 0° C. and dried over P₂O₅ to yield N-pyridinium sulfonic acid (2.42 g) as a white solid, m.p. 128-130° C. m/z (MALDI-TOF) 161.1 [(M+H)⁺ requires 161.2].

### EXAMPLE 12

### Synthesis of 2,4-disulfonic-1,3,5,8-tetramethylporphyrin-6,7-dipropanoic acid-Deuteroporphyrin IX-2,4-disulfonic acid (DSA) 11

Following the method of Smith, 1999 supra; 1976 supra, deuteroporphyrin IX 5 (5.0 mg, 0.0098 mmol) was mixed thoroughly with N-pyridinium sulfonic acid (75.0 mg, mmol) and the mixture fused for 30 min at 165° C., cooled and dissolved in a small amount of dichloromethane (5 ml) and water (5 ml). On standing in an ice-salt bath, deuteroporphyrin IX-2,4-disulfonic acid 11 crystallized as a purple solid. The solid was filtered and the solvent evaporated off to yield deuteroporphyrin IX-2,4-disulfonic acid 11 (4.2 mg, 63.6%) as a purple solid, m.p.>300° C., λₘₐₓ (H₂0)/nm 401 (log ε 5.25), 507 (4.30), 542 (4.24), 569 (4.23), 617 (4.11); m/z (MALDI-TOF) 671.3 [(M+H)⁺ requires 671.7].

### EXAMPLE 13

### Biological Testing

### Preparation of Buffers

### Coating Buffer

The coating buffer was a bicarbonate buffer (0.1 M) containing sodium hydrogen carbonate (50 mM), sodium chloride (137 mM) and sodium azide (10 mM); at pH 9.0.

### Blocking/Washing Buffer (PBS)

The blocking/washing buffer was 1× phosphate-buffered saline containing sodium chloride (137 mM), anhydrous disodium hydrogen phosphate (8.1 mM), potassium chloride (2.7 mM), potassium dihydrogen phosphate (1.5 mM) and sodium azide (10 mM) with Tween-20 (0.1%, v/v 1 ml/L); at pH 7.4.

### Binding Buffer

The binding buffer was an acetate buffer (0.05 mM) containing sodium chloride (150 mM) and sodium azide (10 mM) with Tween-20 (0.1%, v/v 1 ml/L); at pH 5.5.

### Detection Buffer

The detection buffer was a 25×AP developing buffer containing Tris (20 mM), magnesium chloride (1 mM) and sodium azide (10 mM); at pH 9.5.

### EXAMPLE 14

### Biological Testing

### Binding Assays

The mixture of mono-metronidazole-substituted adducts 20 and 21 and heme (both at 5 μg/ml) were coated on the plastic well surfaces of a 96-well plate in coating buffer at pH 9.0. After washing off unbound porphyrin 20 and 21 or heme with Tris/Tween-20 buffer pH 7.5 (TBS), the plates were blocked with TBS for 30 min. Dilutions of rHA2 in binding buffer at pH 5.5 were incubated in quadruplicate with coated porphyrin 20 and 21 or heme for 1 h 30 min at 37° C. before washing with TBS. Primary mouse 5A1 mAb (0.5 μg/ml) was applied in TBS and the plate was incubated for 1 h at 37° C. before washing with TBS. Secondary goat anti-mouse Ab conjugated with Alkaline Phosphatase (AP) was applied in TBS and the plate was incubated for 1 h at 37° C. before washing with TBS. The substrate, para-nitrophenylphosphate (p-NPP), was applied in detection buffer at pH 9.5 and the plate was incubated for 1 h at 37° C. and the AP activity on the dephosphorylation of p-NPP to p-NP was then monitored on a Bio-rad Benchmark plate reader at 405 nm at pre-programmed intervals (absorbance maximum of 3.0 ELISA units). Apparent dissociation constants were calculated from best-fit KD₅₀ binding curves.

### EXAMPLE 15

### Biological Testing

### Growth Inhibition Assays

Cultivation and biological testing were carried out on P. gingivalis ATCC 33277 cells and the cultures were anaerobically grown at 37° C. in an atmosphere containing CO₂ (5%), H₂ (10%) and N₂ (85%). The cultures were inoculated with heme, menadione and either DMSO, DPIX 5, metronidazole, DPIX 5+ metronidazole or adduct 20 and 21. In a 96-well polystyrene plate divided into four sections, a series of dilutions of the mixture of the mono-adducts 20 and 21, metronidazole, DPIX 5 and DMSO were made up. These were transferred into test-tubes in quadruplicate and the P. gingivalis cells were added in triplicate leaving one test-tube as a blank control free of P. gingivalis cells. The growth of the P. gingivalis cells were monitored over 3 days (72 h) and growth was noted by the turbidity of the medium and recorded as the optical density.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any to or more of said steps or features.

### BIBLIOGRAPHY

Adler, et al. Bioinorg Chem 1977 7:187-188
Adler, et al. Inorg Nucl Chem 32:2443-2445, 1970
Albandar Periodontol 29:177-206, 2002
Amano et al. S Infect Immun 66:2072-2077, 1998
Arnoux et al. Proceedings of the National Academy of Science 91: 9876-9888, 1994
Cannon J Pharmaceut Sci 83:435-440, 1993
Clerehugh Community Dental Health 10:19-28, 1993
DeCarlo et al J Bacteriol 181:3784-3791, 1999
Edwards et al. Janssen Research Foundation Series 2:673-676, 1980
Ghayoumi, N. Journal of the Western Society of Periodontology/Periodontal abstracts 49:37-40, 2001
Heasman et al. The Periodontium and Orthodontics in Health and Disease; Oxford University Press: Oxford, 1996
Hunter et al. J Porphyrins Phthalocyanines 6:774-782, 2002
Janson T. R. a. K., J. J. J Mag Reson 6:209-220, 1972
Kenney et al. J Periodontol 40:630-633, 1969
Milgrom, L. R. Polyhedron 4:1661-1664, 1985
Mitchell, D. A. J Clin Periodontol 11:145-158, 1984
Mukherjee, S. J. Periodontol 56:22-27, 1985
Naj di, S., B. Sc. (Hons.) Thesis, University of Sydney, 2000
Novaes et al. A C R J Antimicrob Chemother 28:151-154, 1991
O'Brien-Simpson et al. Current Protein and Peptide Science 4:409-426, 2003
Olive, P. L. Brit J Cancer 40:94-104, 1979
Oliver et al. J Periodontol 69:269-278, 1998
Paramaesvaran et al. J Bacteriol 185:2528-2537, 2003
Passler, R. a. N., H. Zahn-, Mund-, und Kieferheilkunde mit Zentralblatt 77:12-16, 1989
Perrin et al. R W L F Purification of Laboratory Chemicals Pergamon Press: Oxford, 1988
Potempa et al. J Periodontol 24:153-192, 2000
Poulet et al. J Clin Periodontol 26:261-263, 1999
Quinn, M. J., B. Sc. (Hons.) Thesis, University of Sydney, 2002
Royappa et al. Langmuir 14:6207-6214, 1998
Sigeti et al. J Infect Dis 148:1083-1089, 1983
Shah, H. N. Biology of the species Porphyromonas gingivalis; CRC Press: Florida, 1993
Slots, J. a. G., R J J Dent Res 63:412-421, 1984
Smith, K. M. J Porphyrins Phthalocyanines 4:319-324, 1999
Smith, K. M. Porphyrins and Metalloporphyrins; Elsevier Scientific Publishing Company: Amsterdam, 1976

1. A targeted molecular agent (TMA) comprising the general Formula (I): 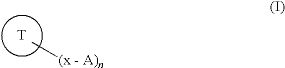
wherein T is a targeting moiety comprising an auxotrophic requirement of a target organism or an analog or derivative of said auxotrophic requirement, x is a chemical linkage entity or linker group, A is an agent required to be targeted to the organism and n is an integer greater than or equal to 1, wherein (x−A), (x−A)₂, (x−A)₃ . . . (x−A)ₙ, may be the same or different and wherein each is independently linked to the targeting moiety via a chemical linkage entity.

2. The TMA of claim 1 wherein the targeting moiety, T is a porphyrin, porphyrin analog or porphyrin derivative.

3. The TMA of claim 2 wherein the porphyrin molecule or analog or derivative is a metal-free porphyrin.

4. The TMA of claim 2 wherein the porphyrin molecule or analog or derivative is a metalloporphyrin.

5. The TMA of claim 1 wherein the agent, A, is selected from the list consisting of Aminoglycosides (including gentamicin, neomycin and streptomycin), Beta-lactams, Penicillins (including Ampicillin, Amoxicillin, Co-amoxiclav and Flucloxacillin), Cephalosporins (including Cefalexin, Cefaclor and Cefuroxime), Chloramphenicol, Cycloserines, lonophores, Glycopeptides, Lincosamides, Macrolides (including Erythromycin and Clarithromycin), Monobactams, Polypeptide antibiotics, Nitroimidazoles (including Metronidazole, Nimorazole and Timidazole), Quinolones (including Ciprofloxacin), Stretogramins, Sulfonamides, Tetracyclines (including Tekacycline, Doxycycline, Oxytetracycline), bambermycin, carbadox, novobiocin, spectinomycin, Clindamycin, Isoniazid, Rifampicin, Trimethoprim (Monoprim) and Vancomycin.

6. The TMA of claim 5 wherein x is an ester linkage.

7. The TMA of claim 5 wherein x is an amide linkage.

8. The TMA of claim 5 wherein x is a urea linkage.

9. The TMA of claim 5 wherein x is a carbamate linkage.

10. The TMA of claim 1 wherein the TMA is targeted to a species of Porphyromonas.

11. The TMA of claim 1 wherein the Porphyromonas is Porphyromonas gingivalis or a related organism selected from the group consisting of Salmonella spp., Serratia spp., Yersinia spp., Klebsiella spp., Vibrio spp., Pseudomas spp., E. coli and Haemophilus spp.

12. A target molecule agent (TMA) comprising metronidazole or a derivative thereof comprising an NO₂ group in a reduced form, said metronidazole linked by a chemical linkage bond to a porphyrin molecule or an analog or derivative thereof.

13. The TMA of claim 12 wherein the TMA is targeted to a species of Porphyromonas.

14. The TMA of claim 12 wherein the Porphyromonas is Porphyromonas gingivalis or a related organism selected from the group consisting of Salmonella spp., Serratia spp., Yersinia spp., Klebsiella spp., Vibrio spp., Pseudomas spp., E. coli and Haemophilus spp.

15. A TMA selected from the group consisting of compounds 20, 21, 39, 40, 41, 42; isomers of compounds 20, 21, 39, 40, 41, 42; monosulfonic acid derivatives of compounds 20, 21, 39, 40, 41, 42; and disulfonic acid derivatives of compounds 20, 21, 39, 40, 41, 42.

16. The isolated TMA of claim 15 wherein the TMA is a 2-sulfonic acid or 4-sulfonic acid derivative of compound 20 or 21.

17. An isolated TMA selected from the group consisting of compound 43, compound 44, isomers of compounds 43 or 44, monosulfonic acid derivatives of compounds 43 or 44 and disulfonic acid derivatives of compounds 43 or 44.

18. A composition comprising the compounds of claim 15 and one or more pharmaceutically acceptable carriers and/or diluents.

19. A method for the prophylaxis or treatment of infection by a microorganism, for which heme is an auxotrophic requirement, in a biological environment from where the microorganism acquires said heme, said method comprising administering to said environment an effective amount of a TMA comprising porphyrin, a porphyrin analog or a porphyrin-like molecule as a targeting moiety for a time and under conditions sufficient to have a microbiocidal or microbiostatic effect on said microorganism.

20. The method of claim 19 wherein the TMA comprises metronidazole or a derivative thereof comprising an NO₂ group in a reduced form, said metronidazole linked via a chemical linkage bond to a porphyrin molecule or an analog or derivative thereof.

21. The method of claim 20 wherein the TMA is selected from the group consisting of compounds 20, 21, 39, 40, 41, 42; isomers of compounds 20, 21, 39, 40, 41, 42; monosulfonic acid derivates of compounds 20, 21, 39, 40, 41, 42 and disulfonic acid derivatives of compounds 20, 21, 39, 40, 41, 42.

22. The method of claim 21 wherein the TMA is a 2-sulfonic acid or 4-sulfonic acid derivative of compound 20 or 21.

23. The method of claim 20 wherein the TMA is selected from the group consisting of compound 43, compound 44, isomers of compounds 43 or 44, monosulfonic acid derivatives of compounds 43 or 44 and disulfonic acid derivatives of compounds 43 or 44.

24. The method of claim 19 wherein the microorganism is Porphyromonas gingivalis.

25. The method of claim 24 wherein the biological environment is the oral cavity.

26. The method of claim 24 wherein the biological environment is selected from the group consisting of pulmonary cavity, vagina, urethra and hoof.

27. A method of enumerating, visualising or localising a subject organism comprising one or more auxotrophic requirements, said method comprising administering to said organism or the environment of said organism a TMA of any one of general Formulae (I), (II) or (III), wherein the targeted agent A is an optically detectable label, and detecting said optically detectable label, wherein detection of the optically detectable label is indicative of the presence, location and/or amount of the organism.

28. A method of manufacture of a medicament for the treatment of periodontal diseases associated with Porphyromonas gingivalis, wherein said medicament contains a TMA of claim 1 or 12 or 15 or 17.
